(19) United States
(12) Patent Application Publication
(10) Pub. No.: US 2026/0130858 A1
(43) Pub. Date: May 14, 2026

(54) LYOPHILIZED SOVATELTIDE-BASED INJECTABLE FORMULATION AND A PROCESS FOR PREPARATION

(71) Applicant: PHARMAZZ, INC., Willowbrook, IL (US)

(72) Inventors: Manish S. LAVHALE, Uttar Pradesh (IN); Anil GULATI, Naperville, IL (US)

(21) Appl. No.: 19/433,400

(22) Filed: Dec. 26, 2025

Related U.S. Application Data
(63) Continuation of application No. PCT/IB2023/062892, filed on Dec. 18, 2023.
Application No. PCT/IB2023/062892 is a continuation-in-part of application No. 18/478,528, filed on Sep. 29, 2023.
Application No. 18/478,528 is a continuation-in-part of application No. 18/343,087, filed on Jun. 28, 2023.

(51) Int. Cl. A61K 9/19; A61K 9/00; A61K 9/08; A61K 38/22; A61K 47/02; A61K 47/12; A61K 47/26

(52) U.S. Cl. CPC A61K 9/19; A61K 9/0019; A61K 9/08; A61K 38/2285; A61K 47/02; A61K 47/12; A61K 47/26

(57) ABSTRACT

A lyophilized Sovateltide-based injectable formulation, comprising sovateltide in the range from about 0.01 to about 0.02% w/w, trisodium citrate dihydrate is present in the range of from about 20 to about 80% w/w and mannitol is present in the range of from about 20 to about 80% w/w. A reconstituted liquid composition comprising sovateltide; trisodium citrate; mannitol and water or 0.9% aqueous sodium chloride solution and process for the preparation of a lyophilized pharmaceutical composition of sovateltide comprising: dissolving sovateltide, trisodium citrate dihydrate and mannitol in water for injection; filter the solution through 0.2μ membrane filter; fill the individual vials up to the target fill volume; and lyophilization of the filled vials.

### CROSS-REFERENCE TO RELATED APPLICATIONS

This application is a continuation-in-part of U.S. Non-Provisional patent application Ser. No. 18/343,087, filed Jun. 28, 2023 and U.S. Non-Provisional patent application Ser. No. 18/478,528 filed Sep. 29, 2023. The contents of which are incorporated by reference herein.

### FIELD OF THE INVENTION

The present invention relates to a lyophilized sovateltide-based injectable pharmaceutical formulation and process for preparation thereof for treatment of various neurological diseases and disorders including but not limited to cerebral ischemic stroke and intracranial haemorrhage. by providing intravenous route of administration and avoiding pain or irritation caused due to conventional procedures.

### BACKGROUND OF THE INVENTION

There are various diseases and disorders that are responsible for discomfort in life along with being fatal for life but have very limited therapeutic solutions. Few of such diseases/disorders includes neurological disorders or diseases including cerebral stroke, Alzheimer's disease, spinal cord injury, cognitive impairment, neurofibromatosis, Huntington's disease, Parkinson's, neonatal hypoxic-ischemic encephalopathy, multi-infarct dementia. Although various drugs have been discovered and synthesized for addressing such diseases and disorders. However, there are still concerns regarding their effectiveness and potential side effects.

Neurological conditions such as cerebral ischemic stroke, intracranial haemorrhage, and brain injury commonly cause cerebral edema. Stroke is one of the most common causes of death and is also an important cause of serious, long-lasting disability. Ischemic stroke caused by arterial occlusion is responsible for most strokes. Various treatment options have been developed for treatment of strokes. Mannitol is one such treatment option as it decreases brain volume by reducing overall water content, blood volume by vasoconstriction, and CSF volume by decreasing water content. Mannitol may also improve cerebral perfusion by reducing viscosity or altering red blood cell rheology.

However, administration of mannitol alone is insufficient to reduce the morbidity and mortality of patients with cerebral stroke. Moreover, monitoring of fluids and electrolytes, serum osmolarity, and renal, cardiac, and pulmonary function needs to be carried out during and following mannitol infusion, which makes the treatment a cumbersome process.

Another treatment option available for treatment of stroke is usage of tissue plasminogen activator (tPA). However, the use of tPA is limited to a short time window of <4.5 h from the onset of symptoms, and it also has a risk of intracranial haemorrhage.

Sovateltide is one such drug having therapeutic potential for treatment of such diseases/disorders, preferably neurological disorders. Sovateltide or succinyl-(glutamyl(9)-alanyl(11,15))-endothelin-1 (8-21) is a compound with 15 amino acid long chain. Sovateltide mimics the properties of a naturally occurring peptide called endothelin-1 (ET-1). ET-1 is known to activate two types of receptors in the body, namely endothelin A (ETARs) and endothelin B (ETBRs). Sovateltide is designed to activate the ETBRs specifically, making it a selective agonist for this receptor type. In fact, the aqueous solution of Sovateltide is susceptible to deterioration during storage.

U.S. Pat. No. 8,623,823 discloses methods of using an ET_{B} receptor agonist, such as IRL-1620, for the treatment of stroke or cerebrovascular accidents. The ET_{B} receptor agonist is used alone or in combination with a second agent useful in the treatment of stroke or other cerebrovascular accidents.

U.S. Pat. No. 10,561,704 discloses compositions and methods for treating neuropsychiatric disorders in vertebrates and humans. More specifically, the present invention provides for use of IRL-1620, an endothelin-B receptor agonist, in appropriate doses to be a neuroprotective and a neuroregenerative agent. Accordingly, in one aspect, the disclosure provides a method of treating a neuropsychiatric disorder comprising administering to a patient in need thereof a therapeutically effective amount of an endothelin-B receptor agonist to treat the neuropsychiatric disorder. In some embodiments, the endothelin-B receptor agonist is co-administered with an additional agent to treat the neuropsychiatric disorder. In some embodiments, the additional agent is selected from the group consisting of an antidepressant, an anti-inflammatory agent, a CNS stimulant, a neuroleptic, and an anti-proliferative agent.

RU2739382C1 discloses FIELD: medicine. SUBSTANCE: invention refers to treating stroke, particularly to a new method for treating stroke involving administration of Imatinib. Imatinib is administered to a patient in dose of 650 to 1600 mg/day on day 1 and in dose of 650 to 1200 mg/day for at least 2 successive consecutive days, preferably for at least 3 successive consecutive days and most preferably for at least 4 successive consecutive days. Imatinib administration for more than 3 days improves the neurological outcome in the post-stroke patients, as well as improves functional independence of the patient. EFFECT: invention improves the treatment of acute ischemic or haemorrhagic stroke, widens the therapeutic window for thrombolysis. RU '382 discloses about usage of Imatinib for treatment of stroke by administering first dose through intravenous route and subsequent doses through oral route. However, since Imatinib is administered through oral route in subsequent doses, the reaction time is more.

U.S. Pat. No. 8,361,459B2 discloses cells derived from postpartum tissue such as the umbilical cord and placenta, and methods for their use to regenerate, repair, and improve neural tissue, and to improve behavior and neurological function in stroke patients. US '459 mentions about the treatment of stroke and other acute neural degenerative disorders by using postpartum-derived cells. However, the method mentioned in the prior art is very specific for stroke and acute neural degenerative disorders, and therefore have narrow range of application.

Conventionally, many compositions and methods have been developed that are capable of treating various neurological conditions. However, since such compositions and methods employ only a single drug, the neurological outcomes of such compositions are not sufficient.

However, such methods either involve the usage of multiple doses of drugs that have high reaction times or have a narrow range of action.

In order to overcome the aforementioned drawbacks, there exists a need in the art to develop a lyophilized sovateltide-based pharmaceutical formulation for the treatment of various neurological conditions such as cerebral ischemic stroke, intracranial haemorrhage that is in lyophilized form in order to prevent deterioration of the formulation and capable of being administered through intravenous route in order to prevent any chances of irritation to the patient that generally occur in conventional treatment procedures.

### OBJECTS OF THE INVENTION

The principal object of the present invention is to overcome the disadvantages of the prior art.

An object of the present invention is to develop a formulation containing Sovateltide in a lyophilized form for the treatment of various neurological diseases and disorders, including but not limited to cerebral ischemic stroke, intracranial haemorrhage that exhibits greater inherent stability compared to the Sovateltide solution.

Another object of the present invention is to develop a formulation that is capable of being administered through an intravenous route or intra-arterial route.

Another object of the present invention is to develop a formulation that is patient-compliant.

Yet another object of the present invention is to develop a formulation that contains Sovateltide in a very small amount, thereby preventing any chances of side-effects due to over-dosing of Sovateltide.

The foregoing and other objects, features, and advantages of the present invention will become readily apparent upon further review of the following detailed description of the preferred embodiment as illustrated in the accompanying drawings.

### SUMMARY OF THE INVENTION

The present invention relates to a lyophilized Sovateltide-based injectable formulation and process for preparation thereof that are administrated intravenously or via the intra-arterial route for treating various neurological disorders and diseases.

According to one embodiment of the present invention, a pharmaceutical formulation for the treatment of cerebral stroke comprises of: i) an endothelin-B receptor analog in a dosing range of 0.00001 mg/kg-1 mg/kg, and ii) a drug in a dosing range of 0.0015 g/kg-5 g/kg.

According to the second embodiment of the present invention, the endothelin-B receptor analog includes, but is not limited to sovateltide, BQ-3020, [Ala^{1,3,11,15}]-endothelin, sarafotoxin S6c, endothelin-3, and the drug includes, but not limited to mannitol and citicoline.

According to an embodiment of the present invention, a lyophilized Sovateltide-based injectable formulation, comprising: i) an active pharmaceutical ingredient in the range of 0.01-0.02% w/w, ii) at least two soluble excipients in the range of 20-80% w/w, and iii) water for injection in the range of 1-2% w/w.

According to the third embodiment of the present invention, a lyophilized Sovateltide-based injectable formulation, comprising: i) sovateltide is present in the range from about 0.01 to about 0.02% w/w; trisodium citrate dihydrate in the range of from about 20 to about 80% w/w and mannitol in the range of from about 20 to about 80% w/w.

According to the fourth embodiment of the present invention, a process for preparation of the lyophilized Sovateltide-based injectable formulation comprises of the following steps: i) Trisodium Citrate Dihydrate dissolved in the water for injection in order to obtain a mixture, ii) the active pharmaceutical ingredient-Sovateltide is added in the mixture, followed by addition of the mannitol in order to obtain a solution, iii) the pH of the solution is checked, for making the volume of the solution, followed by sterile filtering the solution and sterile filling the filtered solution in vials, and iv) the plugs on the vials are half stoppered, followed by loading the vials in freeze dryer for lyophilization to obtain the lyophilized Sovateltide-based injectable formulation.

While the invention has been described and shown with particular reference to the preferred embodiment, it will be apparent that variations might be possible that would fall within the scope of the present invention.

### BRIEF DESCRIPTION OF THE DRAWINGS

These and other features, aspects, and advantages of the present invention will become better understood with regard to the following description, appended claims, and accompanying drawings where:

FIG. 1 illustrates a graphical representation of modified Rankin scale of patients treated with control and sovateltide along with mannitol;

FIG. 2 illustrates a graphical representation of number of cerebral ischemic stroke patients treated with control and sovateltide along with mannitol and having a modified Rankin Scale of 0-2 at 90 days post-randomization;

FIG. 3 illustrates a graphical representation of number of cerebral ischemic stroke patients treated with control and sovateltide along with mannitol and having improvement of 2 or more modified Rankin Scale from baseline at 90 days post-randomization;

FIG. 4 illustrates a graphical representation of modified Rankin scale of patients treated with control and sovateltide along with citicoline;

FIG. 5 illustrates a graphical representation of number of cerebral ischemic stroke patients treated with control and sovateltide along with citicoline and having a modified Rankin Scale of 0-2 at 90 days post-randomization;

FIG. 6 illustrates a graphical representation of number of cerebral ischemic stroke patients treated with control and sovateltide along with citicoline and having improvement of 2 or more modified Rankin Scale from baseline at 90 days post-randomization;

FIG. 7 illustrates a graphical of modified Rankin scale of patients treated with control and sovateltide along with cerebrolysin;

FIG. 8 illustrates a graphical representation of number of cerebral ischemic stroke patients treated with control and sovateltide along with cerebrolysin and having a modified Rankin Scale of 0-2 at 90 days post-randomization;

FIG. 9 illustrates a graphical representation of number of cerebral ischemic stroke patients treated with control and sovateltide along with cerebrolysin and having improvement of 2 or more modified Rankin Scale from baseline at 90 days post-randomization;

FIG. 10 illustrates a graphical representation of modified Rankin scale of patients treated with control and sovateltide along with thrombolytics;

FIG. 11 illustrates a graphical representation of number of cerebral ischemic stroke patients treated with control and sovateltide along with thrombolytics and having a modified Rankin Scale of 0-2 at 90 days post-randomization;

FIG. 12 illustrates a graphical representation of number of cerebral ischemic stroke patients treated with control and sovateltide along with thrombolytics and having improvement of 2 or more modified Rankin Scale from baseline at 90 days post-randomization; and

FIG. 13 illustrates a flow chart of a process of developing lyophilized Sovateltide-based injectable formulation.

### DETAILED DESCRIPTION OF THE INVENTION

The following description includes the preferred best mode of one embodiment of the present invention. It will be clear from this description of the invention that the invention is not limited to these illustrated embodiments but that the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a,” “an,” and “the” designate both the singular and the plural, unless expressly stated to designate the singular only.

As used herein, the term “Pharmaceutical dosage forms” refer to specific forms or preparations in which drugs or pharmaceutical substances are formulated and administered to patients for therapeutic purposes. Such dosage forms may vary in their physical state, such as solid, liquid or semi-solid, and their route of administration, such as oral, topical, intravenous, or inhalation.

As used herein, the term “freeze-dried or lyophilized” powder or cake or preparation refers to any solid substance that has undergone the process of lyophilization or freeze-drying of aqueous solution. Ideally, a lyophilized preparation is obtained by freeze-drying a solution made up of aqueous solvents.

As used herein, the term “Stable pharmaceutical composition” refers to a formulation of a drug or active ingredient that maintains its physical, chemical, and therapeutic properties over a prolonged period, under appropriate storage conditions.

The present invention is pharmaceutical formulation for treatment of neurological disorders that includes an endothelin-B receptor analog and a drug, wherein the analog shows synergistic effect with the drug, resulting in enhanced therapeutic action of the formulation against various neurological conditions such as stroke.

The endothelin-B receptor analog discloses herein is selected from a group comprising sovateltide, BQ-3020, [Ala^{1,3,11,15}]-endothelin, sarafotoxin S6c, endothelin-3. The drug disclosed herein includes, but not limited to mannitol and citicoline.

Sovateltide as an active ingredient, including a liquid injection having a solvent, that comprises at least one soluble excipient, wherein the soluble excipient includes mannitol, Trisodium citrate, Citric Acid anhydrous, Dibasic sodium citrate, Dibasic sodium Phosphate, Sodium Chloride, Hydroxypropyl cyclodextrin, where the soluble excipient serves multiple purposes of functioning as an osmolarity adjusting agent, pH modifier, and solubility enhancer.

The osmolarity adjusting agent (buffering agent), pH modifier, and solubility enhancer are selected from the group consisting of sodium chloride, Trisodium citrate, Citric Acid anhydrous, Dibasic sodium citrate, Dibasic sodium Phosphate, Hydroxypropyl β-cyclodextrin, and mannitol and mixture thereof.

The freeze-drying filler is selected from the group consisting of sodium chloride, Trisodium citrate, Citric Acid anhydrous, Dibasic sodium citrate, Dibasic sodium Phosphate, Hydroxypropyl β-cyclodextrin, and mannitol and mixture thereof

In one aspect, the present invention relates to a lyophilized pharmaceutical composition comprising: a) sovateltide; b) trisodium citrate; and c) mannitol. In one embodiment, a) sovateltide is present in the range from about 0.01% to about 0.02% w/w; b) trisodium citrate is present in the range from about 20% to about 80% w/w; and c) mannitol is present in the range from about 20% to about 80% w/w. In yet another embodiment, a) sovateltide is present at about 30 μg; b) trisodium citrate is present at about 50 mg; and c) mannitol is present at about 160 mg.

In specific, the present invention recites of a lyophilized Sovateltide-based injectable formulation, comprising: i) sovateltide is present in the range from about 0.01 to about 0.02% w/w; trisodium citrate dihydrate in the range of from about 20 to about 80% w/w and mannitol in the range of from about 20 to about 80% w/w.

The formulation herein is prepared as a lyophilized formulation and is capable of being administered through intravenous or intra-arterial route.

The formulation is administered through a catheter with or without adjunctive mechanical methods or during the procedure of embolectomy or thrombectomy.

The formulation is prepared in the form of nanoparticle, gel or hydrogel, nano-emulsion, microparticle, colloidal suspension, sterile suspension, solution, aerosol, and powder.

The formulation comprises a biodegradable polymer, wherein the biodegradable polymer is poly(lactic-co-glycolic acid) (PLGA) or pegylated PLGA (PEG-PLGA).

The formulation includes additives, including but not limited to polyvinyl alcohol (PVA) or other known nanoparticle stabilizers.

The lyophilized pharmaceutical composition comprises about 30 μg of Sovateltide; about 50 mg of trisodium citrate dihydrate; and about 160 mg of mannitol.

The lyophilized pharmaceutical composition further comprises total impurities not more than 2%; or any unspecified impurities not more than 0.5%; or D-His-sovateltide impurity not more than 1.0%.

The lyophilized pharmaceutical composition comprises: total impurities not more than 0.7%; or any unspecified impurities not more than 0.15%; or D-His-sovateltide impurity not more than 0.22%. The composition is reconstituted by 0.9% sodium chloride solution and by water for injection.

A method of treating a neurological disorder or disease comprising administering the lyophilized pharmaceutical composition to a patient in need thereof.

The neurological disorder or disease is selected from cerebral stroke, Alzheimer's disease, spinal cord injury, cognitive impairment, neurofibromatosis, Huntington's disease, Parkinson's, neonatal hypoxic-ischemic encephalopathy, traumatic brain injury, and multi-infarct dementia.

A reconstituted liquid composition is prepared that comprises sovateltide; trisodium citrate; mannitol; and water or 0.9% aqueous sodium chloride solution. In one embodiment, wherein sovateltide, trisodium citrate and mannitol in the reconstituted liquid composition are provided as a lyophilized powder.

The liquid pharmaceutical composition wherein the sovateltide is present in the range from about 0.01 to about 0.02% w/w; trisodium citrate dihydrate is present in the range of from about 20 to about 80% w/w and mannitol is present in the range of from about 20 to about 80% w/w.

Furthermore, sovateltide is present at about 30 g; trisodium citrate is present at about 50 mg; and mannitol is present at about 160 mg in the liquid composition.

In the liquid composition sovateltide is present at 6 μg/ml strength.

The liquid composition further comprises: total impurities not more than 2%; or any unspecified impurities not more than 0.5%; or D-His-sovateltide impurity not more than 1.0%.

The liquid composition further comprises: total impurities not more than 0.7%; or any unspecified impurities not more than 0.15%; or D-His-sovateltide impurity not more than 0.22%.

The osmolality is between 240 to 310 mOsm/L for the reconstituted liquid composition and preferably of about 286 mOsm/L. The pH of the liquid composition is between 7.0 to 8.5.

The present invention also includes a kit or a co-pack comprising a vial containing lyophilized pharmaceutical composition of sovateltide; and a ampoule/vials containing 0.9% sodium chloride aqueous solution or water for injection. The kit or co-pack of the present invention, wherein the vial containing lyophilized pharmaceutical composition of sovateltide comprises: about 30 μg of sovateltide; about 50 mg of trisodium citrate dihydrate; and about 160 mg of mannitol.

The kit or co-pack comprises multiple vials containing lyophilized pharmaceutical composition of sovateltide and multiple ampoules/vials of 0.9% sodium chloride aqueous solution or water for injection.

Furthermore, the kit or co-pack contains 3 vials of lyophilized pharmaceutical composition of sovateltide and 3 ampoules/vials of 0.9% sodium chloride aqueous solution or water for injection.

The present invention relates to a lyophilized Sovateltide-based injectable formulation and method for preparation thereof that is beneficial for the treatment of various neurological disorders and diseases without causing any side effects to the human body due to usage of less amount of Sovateltide in the formulation.

According to an embodiment of the present invention, a lyophilized Sovateltide-based injectable formulation, comprising: i) an active pharmaceutical ingredient in the range of 0.01-0.02% w/w, ii) at least two soluble excipient in the range of 20-80% w/w, and iii) water for injection in the range of 1-2% w/w.

In one aspect, the present invention relates to a process for the preparation of a lyophilized pharmaceutical composition of sovateltide comprising: i) dissolving sovateltide, trisodium citrate dihydrate and mannitol in water for injection; ii) filter the solution through 0.2μ membrane filter; iii) fill the individual vials up to the target fill volume; and iv) lyophilization of the filled vials. In one embodiment, the solution in step i) is stirred at 300-350 rpm. In yet another embodiment, the pH of the solution in step i) is adjusted to pH of about 7.5 to 8.5.

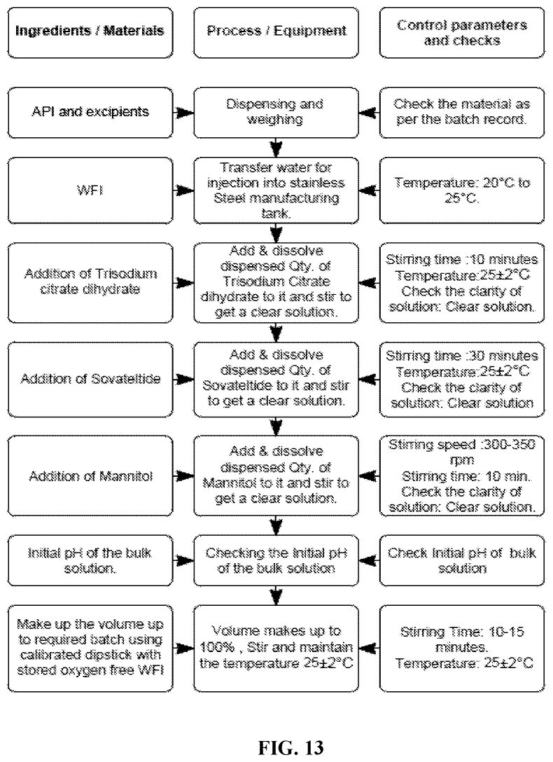

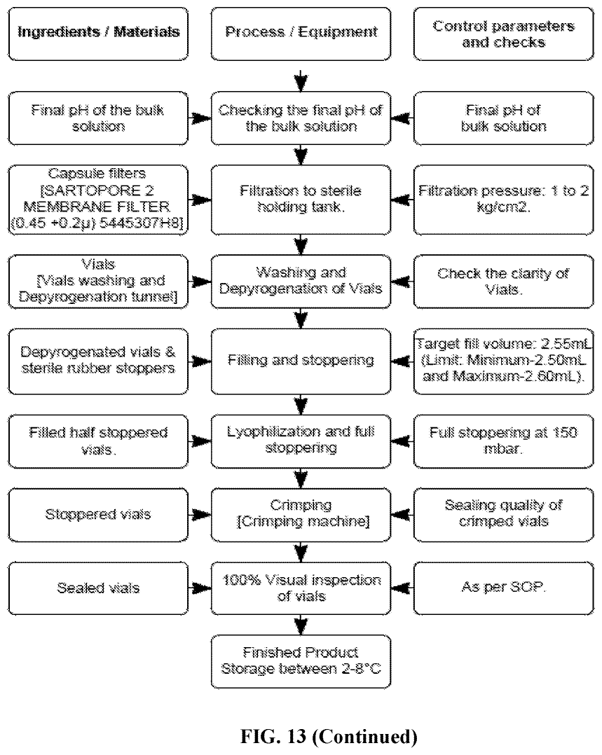

The process for preparation of the lyophilized Sovateltide-based injectable formulation comprises of following steps: i) dissolving Trisodium Citrate Dihydrate in the water for injection in order to obtain a mixture, ii) adding the active pharmaceutical ingredient-Sovateltide in the mixture, followed by addition of the mannitol in order to obtain a solution, iii) checking the pH of the solution, for making the volume of the solution, followed by sterile filtering the solution and sterile filling the filtered solution in vials, and iv) half stoppering of plugs on the vials followed by loading the vials in freeze dryer for lyophilization to obtain the formulation (as illustrated in FIG. 13).

The process is carried at about 20° C. to 30° C.

The process wherein the pH of the solution is adjusted to pH of about 7.5 to 8.5.

In an embodiment, the present invention comprises Sovateltide as an active ingredient, including a liquid injection having a solvent, that comprises at least one soluble excipient, wherein the soluble excipient includes mannitol, Trisodium citrate, Citric Acid anhydrous, Dibasic sodium citrate, Dibasic sodium Phosphate, Sodium Chloride, Hydroxypropyl cyclodextrin, where the soluble excipient serves multiple purposes of functioning as an osmolarity adjusting agent, pH modifier, and solubility enhancer.

In another embodiment, the osmolarity adjusting agent (buffering agent), pH modifier, and solubility enhancer are selected from the group consisting of sodium chloride, Trisodium citrate, Citric Acid anhydrous, Dibasic sodium citrate, Dibasic sodium Phosphate, Hydroxypropyl β-cyclodextrin, and mannitol and mixture thereof.

In another embodiment, the freeze-drying filler is selected from the group consisting of sodium chloride, Trisodium citrate, Citric Acid anhydrous, Dibasic sodium citrate, Dibasic sodium Phosphate, Hydroxypropyl β-cyclodextrin, and mannitol and mixture thereof.

The presently disclosed subject matter is further illustrated by the following specific but not-limiting examples.

The pharmaceutical formulation for treatment of cerebral stroke comprises of 0.0003 mg/kg of sovateltide and 0.75 to 1.25 g/kg of mannitol or 0.0003 mg/kg of sovateltide and 15 to 30 mg/kg of citicoline.

For administration of the proposed formulation comprising mannitol and sovateltide, mannitol infusion is followed by administration of sovateltide. Similarly, for administration of the proposed formulation comprising citicoline and sovateltide, citicoline infusion is followed by administration of sovateltide.

According to another embodiment of the present invention, sovateltide is administered before, after, or simultaneously with mannitol or citicoline.

Accordingly, to another embodiment of the present invention, an example of treatment with mannitol and sovateltide is as follows: the treatment is initiated upon admission of a patient with a neurological deficit, and mannitol solution is administered intravenously. The dose may range from 0.1 g/kg to 5.0 g/kg daily. The average dose of mannitol during the 24 hours of administration is about 0.75 to 1.25 g/kg, and the duration of mannitol treatment can continue for about 3 to 10 days, with most patients receiving mannitol for about six days. Another way to administer mannitol is to infuse 100 ml of 20% mannitol given every four hours for five to six days. Sovateltide is administered in the dose of 0.3 μg/kg as an intravenous bolus over one minute at multiple intervals.

Accordingly, to another embodiment of the present invention, an example of treatment with citicoline and sovateltide is as follows: the treatment is initiated within twenty-four hours after the onset of symptoms of neurological deficits. The citicoline administration is done in a dose ranging from 1.5 mg/kg to 75 mg/kg intravenously every twelve hours during the first three to six days and then orally for six weeks in a dose of 1000 grams every twelve hours. Sovateltide is to be administered in the dose of 0.3 μg/kg as an intravenous bolus over one minute at multiple intervals.

A prospective, multicentric, randomized phase III study was conducted in acute cerebral ischemic stroke patients aged 18 years through 78 years. Patients with radiologic confirmation of ischemic stroke could be enrolled if presenting up to 24 hours after the onset of symptoms and with a modified Rankin Score (mRS) of 3 to 4 and an NIHSS (National Institute of Health Stroke Scale) score of greater than 5. Patients with intracranial haemorrhage and those receiving endovascular therapy were excluded.

Patients were randomized in a 1:1 ratio, 80 patients to the sovateltide group and 78 patients to the control (saline) group. Every patient received the standard of care (SOC) and was followed for 90 days. The study drug (normal saline or sovateltide (0.3 μg/kg) was administered in three doses administered as an intravenous bolus over 1 minute at an interval of 3 hours±1 hour on day 1, day 3, and day 6 (total sovateltide dose of 0.9 μg/kg/day).

The primary objectives were to determine the neurological outcome based on modified Rankin Scale (mRS) score, National Institute of Health Stroke Scale (NIHSS) score, and Barthel Index (BI) scale score from day 1 through day 90. In addition, quality-of-Life was measured by EuroQol-EQ-5D (EQ-5D) and stroke-specific quality-of-life (SSQoL) scores at 60 days and 90 days of treatment.

A total of 158 patients with acute cerebral ischemic stroke were enrolled in this test, of which 137 completed 90-day follow-up. Patients received saline (n=70; 45 male and 25 female) or sovateltide (n=67; 45 male and 22 female) within 24 hours of the onset of stroke. Patients received the investigational drug (mannitol or citicoline) at 16.85±0.74 and 17.40±0.67 hours (mean±SEM, p=0.583) of stroke onset in control and sovateltide cohorts, respectively. The baseline characteristics and SOC in both cohorts were similar. Alberta Stroke Program Early Computed Tomographic Score (ASPECTS) mean value was similar in the control (7.44) and sovateltide (7.61) groups, indicating that the extent of infarction was similar in both the groups.

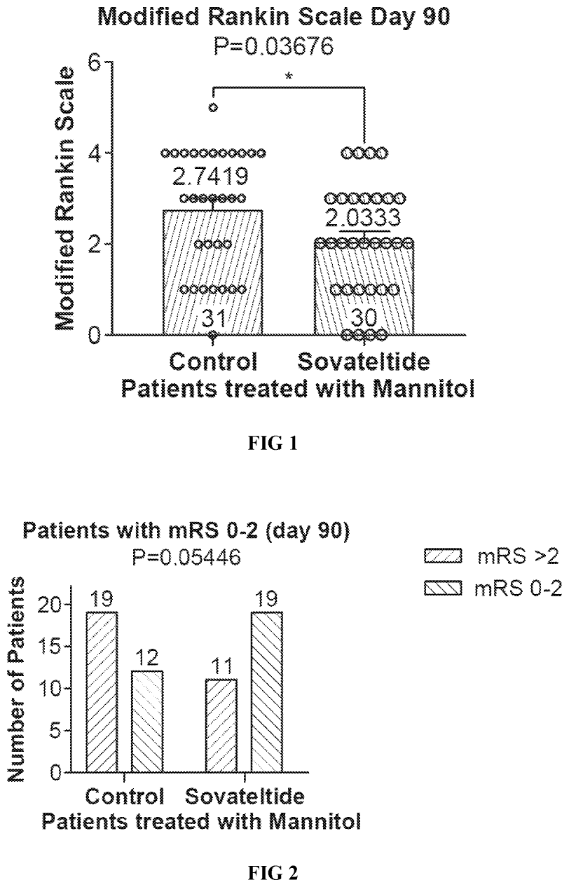

The modified Rankin Scale at post-randomization day 90 of control and sovateltide cerebral ischemic stroke patients (N=61) treated with mannitol was determined in 31 patients of the control group and 30 patients in the sovateltide group. Referring to FIG. 1 and table 1, a graphical and a tabular representation of the modified Rankin scale of patients treated with control and sovateltide along with mannitol are illustrated, respectively. It was found that patients treated with mannitol and sovateltide had significantly lower modified Rankin Scale compared to those that received mannitol.

TABLE 1
Modified Rankin scale of patients treated
with control and sovateltide along with mannitol
Modified Rankin Scale Control vs Sovateltide Day 90 Mannitol

| | |
|---|---|
| Column B | Sovateltide |
| vs. | vs. |
| Column A | Control |

Unpaired t test

| | |
|---|---|
| P value | 0.0368 |
| P value summary | * |
| Significantly different | Yes |
| (P < 0.05)? | |
| One- or two-tailed P value? | Two-tailed |
| t, df | t = 2.137, df = 59 |
| Mean of column A | 2.742 |
| Mean of column B | 2.033 |
| Difference between means | −0.7086 ± 0.3316 |
| (B − A) ± SEM | |
| 95% confidence interval | −1.372 to −0.04509 |
| R squared (eta squared) | 0.07184 |

Data analyzed

| | |
|---|---|
| Sample size, column A | 31 |
| Sample size, column B | 30 |

Referring to FIG. 2 and table 2, a graphical representation and a tabular representation of number of cerebral ischemic stroke patients treated with control and sovateltide along with mannitol and having a modified Rankin Scale of 0-2 at 90 days post-randomization are illustrated, respectively. The number of cerebral ischemic stroke patients with a modified Rankin Scale of 0-2 at 90 days post-randomization was significantly more in those treated with mannitol and an endothelin-B agonist, sovateltide, compared to those with mannitol treatment.

TABLE 2
Number of cerebral ischemic stroke patients treated with
control and sovateltide along with mannitol and having a
modified Rankin Scale of 0-2 at 90 days post-randomization
Number of Patients with mRS of 0-2 day 90 Mannitol
P value and statistical significance

| Test | Chi-square | |
|---|---|---|
| Chi-square, df | 3.699, 1 | |
| Z | 1.923 | |
| P value | 0.0545 | |
| P value summary | ns | |
| One- or two-sided | Two-sided | |
| Statistically significant | No | |
| (P < 0.05)? | | |
| Effect size | Value | 95% CI |
| Relative Risk | 1.672 | 0.9905 to 2.963 |
| Reciprocal of relative risk | 0.5982 | 0.3375 to 1.010 |
| Odds ratio | 2.735 | 0.9381 to 7.734 |
| Reciprocal of odds ratio | 0.3657 | 0.1293 to 1.066 |

Methods used to compute CIs

| | | |
|---|---|---|
| Relative Risk | Koopman | |
| | asymptotic score | |
| Odds ratio | Baptista-Pike | |
| Data analyzed | mRS > 2 | mRS 0-2 |
| Control | 19 | 12 |
| Sovateltide | 11 | 19 |
| Total | 30 | 31 |
| Percentage of row total | mRS > 2 | mRS 0-2 |
| Control | 61.29% | 38.71% |
| Sovateltide | 36.67% | 63.33% |

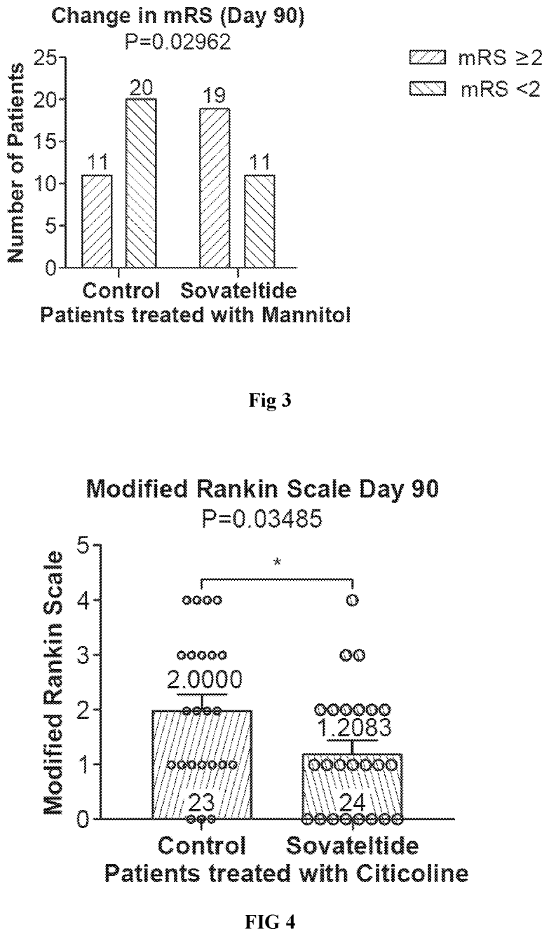

Referring to FIG. 3 and table 3, a graphical representation and a tabular representation of number of cerebral ischemic stroke patients treated with control and sovateltide along with mannitol and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was significantly more in the group treated with mannitol and an endothelin-B agonist, sovateltide compared to the group treated with mannitol. Therefore, sovateltide improved the neurological outcome of mannitol.

TABLE 3
Number of cerebral ischemic stroke patients treated with control
and sovateltide along with mannitol and having improvement of 2 or more
modified Rankin Scale from baseline at 90 days post-randomization
Change in mRS of 2 or more from day 1 to day 90 Mannitol
P value and statistical significance

| Test | Chi-square | |
|---|---|---|
| Chi-square, df | 4.731, 1 | |
| Z | 2.175 | |
| P value | 0.0296 | |
| P value summary | * | |
| One- or two-sided | Two-sided | |
| Statistically significant | Yes | |
| (P < 0.05)? | | |
| Effect size | Value | 95% CI |
| Relative Risk | 0.5603 | 0.3163 to 0.9456 |
| Reciprocal of relative risk | 1.785 | 1.058 to 3.161 |
| Odds ratio | 0.3184 | 0.1093 to 0.9381 |
| Reciprocal of odds ratio | 3.140 | 1.066 to 9.149 |

Methods used to compute CIs

| | | |
|---|---|---|
| Relative Risk | Koopman | |
| | asymptotic score | |
| Odds ratio | Baptista-Pike | |
| Data analyzed | mRS ≥ 2 | mRS < 2 |
| Control | 11 | 20 |
| Sovateltide | 19 | 11 |
| Total | 30 | 31 |
| Percentage of row total | mRS ≥ 2 | mRS < 2 |
| Control | 35.48% | 64.52% |
| Sovateltide | 63.33% | 36.67% |

The modified Rankin Scale at post-randomization day 90 of control and sovateltide cerebral ischemic stroke patients (N=47) treated with citicoline was determined in 23 patients of the control group and 24 patients in the sovateltide group. Referring to FIG. 4 and table 4, a graphical and a tabular representation of modified Rankin scale of patients treated with control and sovateltide along with citicoline are illustrated, respectively. It was found that patients treated with citicoline and an endothelin-B agonist, sovateltide, had significantly better neurological outcomes with significantly lower modified Rankin Scale compared to those that received citicoline.

TABLE 4
Modified Rankin scale of patients treated with
control and sovateltide along with citicoline
Modified Rankin Scale Control vs Sovateltide Day 90 Citicoline

| | |
|---|---|
| Column B | Sovateltide |
| vs. | vs. |
| Column A | Control |

Unpaired t test

| | |
|---|---|
| P value | 0.0348 |
| P value summary | * |
| Significantly different | Yes |
| (P < 0.05)? | |
| One- or two-tailed P value? | Two-tailed |
| t, df | t = 2.176, df = 45 |
| Mean of column A | 2.000 |
| Mean of column B | 1.208 |
| Difference between means | −0.7917 ± 0.3638 |
| (B − A) ± SEM | |
| 95% confidence interval | −1.524 to −0.05888 |
| R squared (eta squared) | 0.09520 |

Data analyzed

| | |
|---|---|
| Sample size, column A | 23 |
| Sample size, column B | 24 |

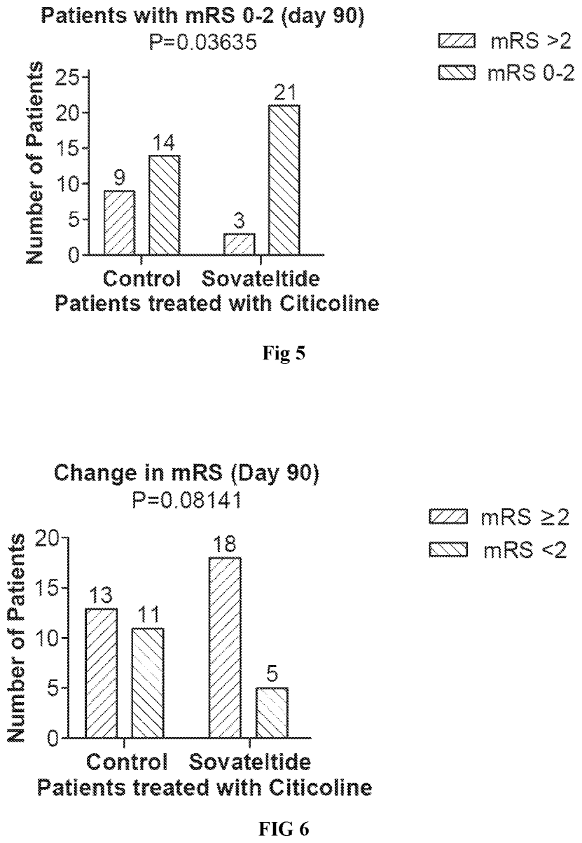

Referring to FIG. 5 and table 5, a graphical representation and a tabular representation of number of cerebral ischemic stroke patients treated with control and sovateltide along with citicoline and having a modified Rankin Scale of 0-2 at 90 days post-randomization are illustrated, respectively. The number of cerebral ischemic stroke patients with a modified Rankin Scale of 0-2 at 90 days post-randomization was significantly more in those treated with citicoline and an endothelin-B agonist, sovateltide compared to those with citicoline treatment.

TABLE 5
Number of cerebral ischemic stroke patients treated with
control and sovateltide along with citicoline and having
a modified Rankin Scale of 0-2 at 90 days post-randomization
Number of Patients with mRS of 0-2 day 90 Citicoline
P value and statistical significance

| Test | Chi-square | |
|---|---|---|
| Chi-square, df | 4.381, 1 | |
| Z | 2.093 | |
| P value | 0.0363 | |
| P value summary | * | |
| One- or two-sided | Two-sided | |
| Statistically significant | Yes | |
| (P < 0.05)? | | |
| Effect size | Value | 95% CI |
| Relative Risk | 3.130 | 1.067 to 9.842 |
| Reciprocal of relative | 0.3194 | 0.1016 to 0.9376 |
| risk | | |
| Odds ratio | 4.500 | 1.058 to 17.02 |
| Reciprocal of odds ratio | 0.2222 | 0.05875 to 0.9455 |

Methods used to compute CIs

| | | |
|---|---|---|
| Relative Risk | Koopman | |
| | asymptotic score | |
| Odds ratio | Baptista-Pike | |
| Data analyzed | mRS > 2 | mRS 0-2 |
| Control | 9 | 14 |
| Sovateltide | 3 | 21 |
| Total | 12 | 35 |
| Percentage of row total | mRS > 2 | mRS 0-2 |
| Control | 39.13% | 60.87% |
| Sovateltide | 12.50% | 87.50% |

Referring to FIG. 6 and table 6, a graphical representation and a tabular representation of number of cerebral ischemic stroke patients treated with control and sovateltide along with citicoline and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was significantly more in the group treated with citicoline and an endothelin-B agonist, sovateltide compared to the group treated with citicoline. Therefore, sovateltide improved the neurological outcome of citicoline.

TABLE 6
Number of cerebral ischemic stroke patients treated
with control and sovateltide along with citicoline
and having improvement of 2 or more modified Rankin
Scale from baseline at 90 days post-randomization
Change in mRS of 2 or more from day 1 to day 90 Citicoline
P value and statistical significance

| Test | Chi-square | |
|---|---|---|
| Chi-square, df | 3.037, 1 | |
| Z | 1.743 | |
| P value | 0.0814 | |
| P value summary | ns | |
| One- or two-sided | Two-sided | |
| Statistically significant | No | |
| (P < 0.05)? | | |
| Effect size | Value | 95% CI |
| Relative Risk | 0.6921 | 0.4326 to 1.049 |
| Reciprocal of relative risk | 1.445 | 0.9535 to 2.311 |
| Odds ratio | 0.3283 | 0.09738 to 1.121 |
| Reciprocal of odds ratio | 3.046 | 0.8924 to 10.27 |

Methods used to compute CIs

| | | |
|---|---|---|
| Relative Risk | Koopman | |
| | asymptotic score | |
| Odds ratio | Baptista-Pike | |
| Data analyzed | mRS ≥ 2 | mRS < 2 |
| Control | 13 | 11 |
| Sovateltide | 18 | 5 |
| Total | 31 | 16 |
| Percentage of row total | mRS ≥ 2 | mRS < 2 |
| Control | 54.17% | 45.83% |
| Sovateltide | 78.26% | 21.74% |

Accordingly to another embodiment of the present invention, a formulation comprising sovateltide and cerebrolysin was also prepared. The modified Rankin Scale at post-randomization day 90 of control and sovateltide cerebral ischemic stroke patients (N=26) treated with cerebrolysin was determined in 14 patients of the control group and 12 patients in the sovateltide group.

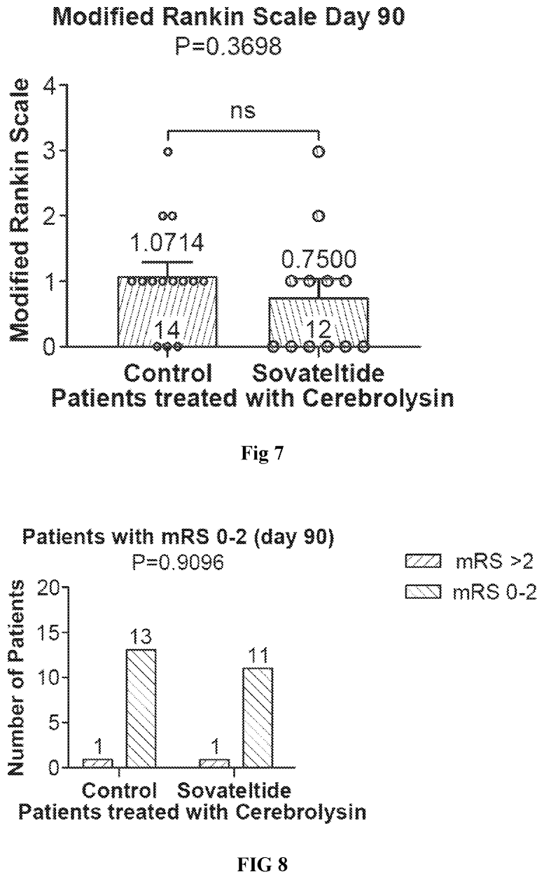

Referring to FIG. 7 and table 7, a graphical and a tabular representation of modified Rankin scale of patients treated with control and sovateltide along with cerebrolysin are illustrated, respectively. It was found that patients treated with cerebrolysin and an endothelin-B agonist, sovateltide, had similar neurological outcomes compared to those that received cerebrolysin.

TABLE 7
Modified Rankin scale of patients treated with
control and sovateltide along with cerebrolysin
Modified Rankin Scale Control vs Sovateltide Day 90 Cerebrolysin

| | |
|---|---|
| Column B | Sovateltide |
| vs. | vs. |
| Column A | Control |

Unpaired t test

| | |
|---|---|
| P value | 0.3698 |
| P value summary | ns |
| Significantly different | No |
| (P < 0.05)? | |
| One- or two-tailed P value? | Two-tailed |
| t, df | t = 0.9140, df = 24 |
| Mean of column A | 1.071 |
| Mean of column B | 0.7500 |
| Difference between means | −0.3214 ± 0.3517 |
| (B − A) ± SEM | |
| 95% confidence interval | −1.047 to 0.4044 |
| R squared (eta squared) | 0.03364 |

Data analyzed

| | |
|---|---|
| Sample size, column A | 14 |
| Sample size, column B | 12 |

Referring to FIG. 8 and table 8, a graphical representation and a tabular representation of number of cerebral ischemic stroke patients treated with control and sovateltide along with cerebrolysin and having a modified Rankin Scale of 0-2 at 90 days post-randomization are illustrated, respectively. The number of cerebral ischemic stroke patients with a modified Rankin Scale of 0-2 at 90 days post-randomization was also similar in those treated with cerebrolysin and an endothelin-B agonist, sovateltide compared to those with cerebrolysin treatment.

TABLE 8
Number of cerebral ischemic stroke patients treated with
control and sovateltide along with cerebrolysin and having
a modified Rankin Scale of 0-2 at 90 days post-randomization
Number of Patients with mRS of 0-2 day 90 Cerebrolysin
P value and statistical significance

| Test | Chi-square | |
|---|---|---|
| Chi-square, df | 0.01290, 1 | |
| Z | 0.1136 | |
| P value | 0.9096 | |
| P value summary | ns | |
| One- or two-sided | Two-sided | |
| Statistically significant | No | |
| (P < 0.05)? | | |
| Effect size | Value | 95% CI |
| Relative Risk | 0.8571 | 0.09552 to 7.748 |
| Reciprocal of relative risk | 1.167 | 0.1291 to 10.47 |
| Odds ratio | 0.8462 | 0.04167 to 17.41 |
| Reciprocal of odds ratio | 1.182 | 0.05745 to 24.00 |

Methods used to compute CIs

| | | |
|---|---|---|
| Relative Risk | Koopman | |
| | asymptotic score | |
| Odds ratio | Baptista-Pike | |
| Data analyzed | mRS > 2 | mRS 0-2 |
| Control | 1 | 13 |
| Sovateltide | 1 | 11 |
| Total | 2 | 24 |
| Percentage of row total | mRS > 2 | mRS 0-2 |
| Control | 7.14% | 92.86% |
| Sovateltide | 8.33% | 91.67% |

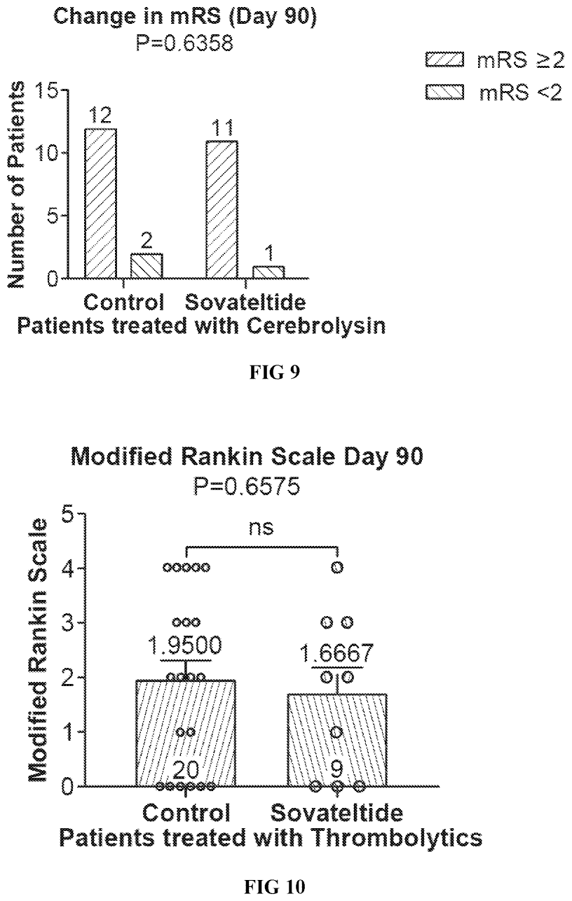

Referring to FIG. 9 and table 9, a graphical representation and a tabular representation of number of cerebral ischemic stroke patients treated with control and sovateltide along with cerebrolysin and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was similar in the group treated with cerebrolysin and an endothelin-B agonist, sovateltide compared to the group treated with cerebrolysin. Therefore, sovateltide did not improve the neurological outcome of cerebrolysin.

TABLE 9
Number of cerebral ischemic stroke patients treated
with control and sovateltide along with cerebrolysin
and having improvement of 2 or more modified Rankin
Scale from baseline at 90 days post-randomization
Change in mRS of 2 or more from day 1 to day 90 Cerebrolysin
P value and statistical significance

| Test | Chi-square | |
|---|---|---|
| Chi-square, df | 0.2243, 1 | |
| Z | 0.4736 | |
| P value | 0.6358 | |
| P value summary | ns | |
| One- or two-sided | Two-sided | |
| Statistically significant | No | |
| (P < 0.05)? | | |
| Effect size | Value | 95% CI |
| Relative Risk | 0.9351 | 0.6441 to 1.362 |
| Reciprocal of relative | 1.069 | 0.7343 to 1.553 |
| risk | | |
| Odds ratio | 0.5455 | 0.03485 to 5.316 |
| Reciprocal of odds | 1.833 | 0.1881 to 28.69 |
| ratio | | |

Methods used to compute CIs

| | | |
|---|---|---|
| Relative Risk | Koopman | |
| | asymptotic score | |
| Odds ratio | Baptista-Pike | |
| Data analyzed | mRS > 2 | mRS < 2 |
| Control | 12 | 2 |
| Sovateltide | 11 | 1 |
| Total | 23 | 3 |
| Percentage of row total | mRS ≥ 2 | mRS < 2 |
| Control | 85.71% | 14.29% |
| Sovateltide | 91.67% | 8.33% |

Accordingly to another embodiment of the present invention, a formulation comprising sovateltide and thrombolytics was also prepared. The modified Rankin Scale at post-randomization day 90 of control and sovateltide cerebral ischemic stroke patients (N=29) treated with thrombolytics was determined in 20 patients of the control group and 9 patients in the sovateltide group.

Referring to FIG. 10 and table 10, a graphical and a tabular representation of modified Rankin scale of patients treated with control and sovateltide along with thrombolytics are illustrated, respectively. It was found that patients treated with thrombolytics and an endothelin-B agonist, sovateltide, had similar neurological outcomes compared to those that received thrombolytics.

TABLE 10
Modified Rankin scale of patients treated with
control and sovateltide along with thrombolytics
Modified Rankin Scale Control vs Sovateltide Day 90 Thrombolytics

| | |
|---|---|
| Column B | Sovateltide |
| vs. | vs. |
| Column A | Control |

Unpaired t test

| | |
|---|---|
| P value | 0.6575 |
| P value summary | ns |
| Significantly different | No |
| (P < 0.05)? | |
| One- or two-tailed P value? | Two-tailed |
| t, df | t = 0.4483, df = 27 |
| Mean of column A | 1.950 |
| Mean of column B | 1.667 |
| Difference between means | −0.2833 ± 0.6321 |
| (B − A) ± SEM | |
| 95% confidence interval | −1.580 to 1.014 |
| R squared (eta squared) | 0.007388 |

Data analyzed

| | |
|---|---|
| Sample size, column A | 20 |
| Sample size, column B | 9 |

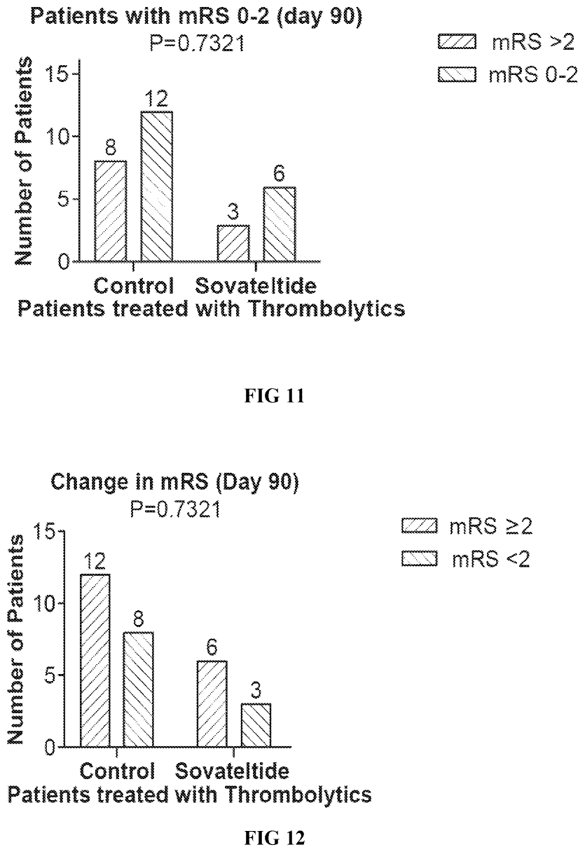

Referring to FIG. 11 and table 11, a graphical representation and a tabular representation of number of cerebral ischemic stroke patients treated with control and sovateltide along with thrombolytics and having a modified Rankin Scale of 0-2 at 90 days post-randomization are illustrated, respectively. The number of cerebral ischemic stroke patients with a modified Rankin Scale of 0-2 at 90 days post-randomization was also similar in those treated with thrombolytics and an endothelin-B agonist, sovateltide compared to those with thrombolytic treatment.

TABLE 11
Number of cerebral ischemic stroke patients treated with
control and sovateltide along with thrombolytics and having
a modified Rankin Scale of 0-2 at 90 days post-randomization
Number of Patients with mRS of 0-2 day 90 Thrombolytics
P value and statistical significance

| Test | Chi-square | |
|---|---|---|
| Chi-square, df | 0.1172, 1 | |
| Z | 0.3423 | |
| P value | 0.7321 | |
| P value summary | ns | |
| One- or two-sided | Two-sided | |
| Statistically significant | No | |
| (P < 0.05)? | | |
| Effect size | Value | 95% CI |
| Relative Risk | 1.200 | 0.4685 to 3.664 |
| Reciprocal of relative risk | 0.8333 | 0.2729 to 2.135 |
| Odds ratio | 1.333 | 0.2496 to 5.929 |
| Reciprocal of odds ratio | 0.7500 | 0.1687 to 4.006 |

Methods used to compute CIs

| | | |
|---|---|---|
| Relative Risk | Koopman | |
| | asymptotic score | |
| Odds ratio | Baptista-Pike | |
| Data analyzed | mRS > 2 | mRS 0-2 |
| Control | 8 | 12 |
| Sovateltide | 3 | 6 |
| Total | 11 | 18 |
| Percentage of row total | mRS > 2 | mRS 0-2 |
| Control | 40.00% | 60.00% |
| Sovateltide | 33.33% | 66.67% |

Referring to FIG. 12 and table 12, a graphical representation and a tabular representation of number of cerebral ischemic stroke patients treated with control and sovateltide along with thrombolytics and having improvement of 2 or more modified Rankin Scale from baseline at 90 days post-randomization are illustrated, respectively. The number of cerebral ischemic stroke patients with an improvement of 2 or more modified Rankin Scale from baseline at 90 days post-randomization was similar in the group treated with thrombolytics and an endothelin-B agonist, sovateltide compared to the group treated with thrombolytics. Therefore, sovateltide did not improve the neurological outcome of thrombolytics.

TABLE 12
Number of cerebral ischemic stroke patients treated with control and
sovateltide along with thrombolytics and having improvement of 2 or more
modified Rankin Scale from baseline at 90 days post-randomization
Change in mRS of 2 or more from day 1 to day 90 Thrombolytics
P value and statistical significance

| Test | Chi-square | |
|---|---|---|
| Chi-square, df | 0.1172, 1 | |
| Z | 0.3423 | |
| P value | 0.7321 | |
| P value summary | ns | |
| One- or two-sided | Two-sided | |
| Statistically significant | No | |
| (P < 0.05)? | | |
| Effect size | Value | 95% CI |
| Relative Risk | 0.9000 | 0.5183 to 1.811 |
| Reciprocal of relative risk | 1.111 | 0.5522 to 1.930 |
| Odds ratio | 0.7500 | 0.1687 to 4.006 |
| Reciprocal of odds ratio | 1.333 | 0.2496 to 5.929 |

Methods used to compute CIs

| | | |
|---|---|---|
| Relative Risk | Koopman | |
| | asymptotic score | |
| Odds ratio | Baptista-Pike | |
| Data analyzed | mRS ≥ 2 | mRS < 2 |
| Control | 12 | 8 |
| Sovateltide | 6 | 3 |
| Total | 18 | 11 |
| Percentage of row total | mRS ≥ 2 | mRS < 2 |
| Control | 60.00% | 40.00% |
| Sovateltide | 66.67% | 33.33% |

Therefore, the combination of sovateltide and mannitol or sovateltide and citicoline is efficient in treatment of neurological conditions as sovateltide is capable of enhancing the efficacy of mannitol and citicoline resulting in improvement of neurological outcomes and may help treat cerebral stroke, intracranial haemorrhage, brain injury, and other neurological conditions causing cerebral edema.

### MORE EXAMPLES

### Example 1

The method for preparation of the lyophilized Sovateltide-based injectable formulation comprises of following steps: i) 4.800 gm of mannitol is dissolved in 75 ml of water for injection in order to obtain a mixture, ii) 0.990 mg of Sovateltide is added in the mixture, followed by addition of 1.500 gm of Trisodium Citrate Dihydrate in order to obtain a solution, iii) the pH of the solution is checked, for making the volume of the solution, followed by filtering the solution and filling the filtered solution in vials, and iv) the plugs on the vials are half stoppered, followed by loading the vials in freeze dryer for lyophilization to obtain the lyophilized Sovateltide-based injectable formulation. Table 1 represents the ingredients along with the composition used in the preparation of lyophilized Sovateltide-based injectable formulation.

TABLE 1
Ingredients along with the composition used in the preparation of
lyophilized Sovateltide-based injectable formulation (Example 1)

| Ingredient | Unit | Quantity used |
|---|---|---|
| Sovateltide | MG | 0.990 |
| Mannitol | GM | 4.800 |
| Trisodium Citrate | GM | 1.500 |
| Water for injection | ML | Q.S. to 75.0 |

### Example 2

A method for preparation of the formulation of: i) 0.3 gm of mannitol is added in 30 ml of water for injection in order to obtain a mixture, ii) 0.990 mg of Sovateltide is added in the mixture, followed by addition of the 1% of Trisodium Citrate Dihydrate in order to obtain a solution, iii) the pH of the solution is checked and adjusted to 7.0, for making the volume of the solution, followed by filtering the solution and filling the filtered solution in vials, and iv) half stoppering of plugs on the vials, followed by loading the vials in freeze dryer for lyophilization to obtain the formulation. Table 2 represents the ingredients along with the composition used in the preparation of lyophilized Sovateltide-based injectable formulation.

TABLE 2
Ingredients along with the composition used in the preparation of
lyophilized Sovateltide-based injectable formulation (Example 2)

| Ingredient | Unit | Quantity used |
|---|---|---|
| Sovateltide | MG | 0.990 |
| Trisodium citrate | GM | 1% solution for pH adjustment |
| Mannitol | GM | 0.3 |
| Water for injection | ML | Q.S. to 30.0 |

### Example 3

The method for preparation of the lyophilized Sovateltide-based injectable formulation is illustrated, which comprises of following steps: i) 0.3 gm of mannitol is added in 30 ml of water for injection in order to obtain a mixture, ii) 0.990 mg of Sovateltide is added in the mixture, followed by addition of 35 mg of 1% of Trisodium Citrate Dihydrate in order to obtain a solution, iii) the pH of the solution is checked and adjusted to pH 5.10 using 1% citric acid solution, for making the volume of the solution, followed by filtering the solution and filling the filtered solution in vials, and iv) half stoppering of plugs on the vials, followed by loading the vials in freeze dryer for lyophilization to obtain the formulation. Table 3 represents the ingredients along with the composition used in the preparation of lyophilized Sovateltide-based injectable formulation.

TABLE 3
Ingredients along with the composition used in the preparation of
lyophilized Sovateltide-based injectable formulation (Example 3)

| Ingredient | Unit | Quantity used |
|---|---|---|
| Sovateltide | MG | 0.990 |
| Trisodium citrate | MG | 35 |
| Mannitol | GM | 0.3 |
| Citric Acid Anhydrous | GM | 1% solution for pH adjustment |
| Water for injection | ML | Q.S. to 30.0 |

### Example 4

The method for preparation of the lyophilized Sovateltide-based injectable formulation comprises of following steps: i) 0.3 gm of mannitol is added in 30 ml of water for injection in order to obtain a mixture, ii) 0.990 mg of Sovateltide is added in the mixture, and dissolve using 0.1% Dibasic sodium Phosphate solution in order to obtain a solution, iii) the pH of the solution is checked and adjusted to pH 7.51, for making the volume of the solution, followed by filtering the solution and filling the filtered solution in vials, and iv) half stoppering of plugs on the vials, followed by loading the vials in freeze dryer for lyophilization to obtain the formulation. Table 4 represents the ingredients along with the composition used in the preparation of lyophilized Sovateltide-based injectable formulation.

TABLE 4
Ingredients along with the composition used in the preparation of
lyophilized Sovateltide-based injectable formulation (Example 4)

| Ingredient | Unit | Quantity used |
|---|---|---|
| Sovateltide | MG | 0.990 |
| Dibasic sodium citrate | GM | 1% solution for pH adjustment |
| Mannitol | GM | 0.3 |
| Water for injection | ML | Q.S. to 30.0 |

### Example 5

The method for preparation of the lyophilized Sovateltide-based injectable formulation comprises of following steps: i) 0.3 gm of mannitol is added in 30 ml of water for injection in order to obtain a mixture, ii) 0.990 mg of Sovateltide is added in the mixture, and dissolve using 9.0 mg Dibasic sodium Phosphate solution in order to obtain a solution, iii) the pH of the solution is checked and adjusted to pH 6.47, using 0.1% citric acid solution for making the volume of the solution, followed by filtering the solution and filling the filtered solution in vials, and iv) half stoppering of plugs on the vials, followed by loading the vials in freeze dryer for lyophilization to obtain the formulation. Table 5 represents the ingredients along with the composition used in the preparation of lyophilized Sovateltide-based injectable formulation.

TABLE 5
Ingredients along with the composition used in the preparation of
lyophilized Sovateltide-based injectable formulation (Example 5)

| Ingredient | Unit | Quantity used |
|---|---|---|
| Sovateltide | MG | 0.990 |
| Dibasic sodium Phosphate | MG | 9.0 |
| Mannitol | GM | 0.3 |
| Citric Acid Anhydrous | GM | 0.1% solution for pH adjustment |
| Water for injection | ML | Q.S. to 30.0 |

### Example 6

The method for preparation of the lyophilized Sovateltide-based injectable formulation comprises of following steps: i) 0.6 gm of mannitol and 60.0 mg of Sodium chloride in 30 ml of water for injection in order to obtain a mixture, ii) 1.980 mg of Sovateltide is added in the mixture, and dissolved using 25.0 mg of Sodium Sulphate in order to obtain a solution, iii) the pH of the solution is checked and adjusted to pH 7.32, using 0.1% citric acid solution for making the volume of the solution, followed by filtering the solution and filling the filtered solution in vials, and iv) half stoppering of plugs on the vials, followed by loading the vials in freeze dryer for lyophilization to obtain the formulation. Table 6 represents the ingredients along with the composition used in the preparation of lyophilized Sovateltide-based injectable formulation.

TABLE 6
Ingredients along with the composition used in the preparation of
lyophilized Sovateltide-based injectable formulation (Example 6)

| Ingredient | Unit | Quantity used |
|---|---|---|
| Sovateltide | MG | 1.980 |
| Sodium Chloride | MG | 60.0 |
| Mannitol | GM | 0.6 |
| Sodium Sulphate | MG | 25.0 |
| Dibasic Sodium Phosphate | GM | 1% solution for pH adjustment |
| Water for injection | ML | Q.S. to 30.0 |

### Example 7

The method for preparation of the lyophilized Sovateltide-based injectable formulation comprises of following steps: i) 0.150 gm of mannitol and 0.150 gm of Hydroxypropyl β-cyclodextrin is added in 30 ml of water for injection in order to obtain a mixture, ii) 0.990 mg of Sovateltide is added in the mixture, and dissolved using 1% of Trisodium Citrate in order to obtain a solution, iii) the pH of the solution is checked and adjusted to 7.10, using 1% solution of Trisodium Citrate Dihydrate for making the volume of the solution, followed by filtering the solution and filling the filtered solution in the vials, and iv) half stoppering of plugs on the vials, followed by loading the vials in freeze dryer for lyophilization to obtain the formulation. Table 7 represents the ingredients along with the composition used in the preparation of lyophilized Sovateltide-based injectable formulation.

TABLE 7
Ingredients along with the composition used in the preparation of
lyophilized Sovateltide-based injectable formulation (Example 7)

| Ingredient | Unit | Quantity used |
|---|---|---|
| Sovateltide | MG | 0.990 |
| Hydroxypropyl β-cyclodextrin | GM | 0.150 |
| Mannitol | GM | 0.150 |
| Trisodium Citrate | GM | 1% solution for pH adjustment |
| Water for injection | ML | Q.S. to 30.0 |

### Example 8

The method for preparation of the lyophilized Sovateltide-based injectable formulation comprises of following steps: i) 0.150 gm of mannitol and 0.150 gm of Hydroxypropyl β-cyclodextrin in 30 ml of water for injection in order to obtain a mixture, ii) 0.990 mg of Sovateltide is added in the mixture, and dissolved using 30 mg of Trisodium Citrate in order to obtain a solution, iii) the pH of the solution is checked and adjusted to pH 5.51 using 1% solution of Citric acid for making the volume of the solution, followed by filtering the solution and filling the filtered solution in vials, and iv) half stoppering of plugs on the vials, followed by loading the vials in freeze dryer for lyophilization to obtain the formulation. Table 8 represents the ingredients along with the composition used in the preparation of lyophilized Sovateltide-based injectable formulation.

TABLE 8
Ingredients along with the composition used in the preparation of
lyophilized Sovateltide-based injectable formulation (Example 8)

| Ingredient | Unit | Quantity used |
|---|---|---|
| Sovateltide | MG | 0.990 |
| Hydroxypropyl β-cyclodextrin | GM | 0.150 |
| Mannitol | GM | 0.150 |
| Citric acid Anhydrous | GM | 1% solution for pH adjustment |
| Trisodium Citrate | MG | 30.0 |
| Water for injection | ML | Q.S. to 30.0 |

### Example 9

The method for preparation of the lyophilized Sovateltide-based injectable formulation is illustrated which comprises of following steps: i) 1.500 gm of mannitol is added in 30 ml of water for injection in order to obtain a mixture, ii) 0.990 mg of Sovateltide is added in the mixture, and dissolve by using 1% solution of Trisodium Citrate Dihydrate in order to obtain a solution, iii) the pH of the solution is checked and adjusted to pH 7.25 by using 1% solution of Trisodium Citrate Dihydrate for making the volume of the solution, followed by filtering the solution and filling the filtered solution in the vials, and iv) half stoppering of plugs on the vials, followed by loading the vials in freeze dryer for lyophilization to obtain the formulation. Table 9 represents the ingredients along with the composition used in the preparation of lyophilized Sovateltide-based injectable formulation.

TABLE 9
Ingredients along with the composition used in the preparation of
lyophilized Sovateltide-based injectable formulation (Example 9)

| Ingredient | Unit | Quantity used |
|---|---|---|
| Sovateltide | MG | 0.990 |
| Mannitol | GM | 1.500 |
| Trisodium Citrate | GM | 1% solution for pH adjustment |
| Water for injection | ML | Q.S. to 30.0 |

### Example 10

The method for preparation of the lyophilized Sovateltide-based injectable formulation comprises of the following steps: i) 0.900 gm of Sodium Chloride is dissolved in 30 ml of water for injection in order to obtain a mixture, ii) 0.900 mg of Sovateltide is added in the mixture, and dissolved by using 1% solution of Trisodium Citrate Dihydrate followed by addition of 0.300 gm of Kollid PF-12 and stir to dissolve to obtain a solution, iii) the pH of the solution is checked and adjusted to pH 6.80 by using 1% solution of Trisodium Citrate Dihydrate for making the volume of the solution, followed by filtering the solution and filling the filtered solution in the vials, and iv) half stoppering of plugs on the vials are, followed by loading the vials in freeze dryer for lyophilization to obtain the formulation. Table 10 represents the ingredients along with the composition used in the preparation of lyophilized Sovateltide-based injectable formulation.

TABLE 10
Ingredients along with the composition used in the preparation of
lyophilized Sovateltide-based injectable formulation (Example 10)

| Ingredient | Unit | Quantity used |
|---|---|---|
| Sovateltide | MG | 0.990 |
| Sodium Chloride | GM | 0.900 |
| Kollidon PF-12 | GM | 0.300 |
| Trisodium Citrate | GM | 1% solution for pH adjustment |
| Water for injection | ML | Q.S. to 30.0 |

### Example 11

The method for preparation of the lyophilized Sovateltide-based injectable formulation is illustrated which comprises of following steps: i) 1.500 gm of Mannitol is dissolved in 30 ml of water for injection in order to obtain a mixture, ii) 0.300 gm of Trisodium Citrate Dihydrate is added in the mixture and stirred to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 11 represents the ingredients along with the composition used in the preparation of lyophilized Sovateltide-based injectable formulation.

TABLE 11
Ingredients along with the composition used in the preparation of
lyophilized Sovateltide-based injectable formulation (Example 11)

| Ingredient | Unit | Quantity used |
|---|---|---|
| Sovateltide | — | — |
| Mannitol | GM | 1.500 |
| Trisodium Citrate | GM | 0.300 |
| Water for injection | ML | Q.S. to 30.0 |

### Example 12

The method for preparation of the lyophilized Sovateltide-based injectable formulation which comprises of following steps: i) 1.500 gm of Mannitol is dissolved in 30 ml of water for injection in order to obtain a mixture, ii) 0.600 gm of Trisodium Citrate Dihydrate is added in the mixture and stirred to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 12 represents the ingredients along with the composition used in the preparation of lyophilized Sovateltide-based injectable formulation.

TABLE 12
Ingredients along with the composition used in the preparation of
lyophilized Sovateltide-based injectable formulation (Example 12)

| Ingredient | Unit | Quantity used |
|---|---|---|
| Sovateltide | — | — |
| Mannitol | GM | 1.500 |
| Trisodium Citrate | GM | 0.600 |
| Water for injection | ML | Q.S. to 30.0 |

### Example 13

The method for preparation of the lyophilized Sovateltide-based injectable formulation comprises of following steps: i) 1.500 gm of Mannitol is dissolved in 30 ml of water for injection in order to obtain a mixture, ii) 0.990 mg of Sovateltide is added in the mixture and dissolved by using 0.900 gm of Trisodium Citrate Dihydrate to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 13 represents the ingredients along with the composition used in the preparation of lyophilized Sovateltide-based injectable formulation.

TABLE 13
Ingredients along with the composition used in the preparation of
lyophilized Sovateltide-based injectable formulation (Example 13)

| Ingredient | Unit | Quantity used |
|---|---|---|
| Sovateltide | MG | 0.990 |
| Mannitol | GM | 1.500 |
| Trisodium Citrate | GM | 0.900 |
| Water for injection | ML | Q.S. to 30.0 |

### Example 14

The method for preparation of the lyophilized Sovateltide-based injectable formulation comprises of following steps: i) 7.500 gm of Mannitol is dissolved in 100 ml of water for injection in order to obtain a mixture, ii) 2.500 gm of Trisodium Citrate Dihydrate is added in the mixture to obtain the solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 14 represents the ingredients along with the composition used in the preparation of lyophilized Sovateltide-based injectable formulation.

TABLE 14
Ingredients along with the composition used in the preparation of
lyophilized Sovateltide-based injectable formulation (Example 14)

| Ingredient | Unit | Quantity used |
|---|---|---|
| Sovateltide | — | — |
| Mannitol | GM | 7.500 |
| Trisodium Citrate | GM | 2.500 |
| Water for injection | ML | Q.S. to 100.0 |

### Example 15

The method for preparation of the lyophilized Sovateltide-based injectable formulation comprises of following steps: i) 7.500 gm of Mannitol is dissolved in 100 ml of water for injection in order to obtain a mixture, ii) 2.500 gm of Trisodium Citrate Dihydrate is added in the mixture and stirred to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 15 represents the ingredients along with the composition used in the preparation of lyophilized Sovateltide-based injectable formulation.

TABLE 15
Ingredients along with the composition used in the preparation of
lyophilized Sovateltide-based injectable formulation (Example 15)

| Ingredient | Unit | Quantity used |
|---|---|---|
| Sovateltide | — | — |
| Mannitol | GM | 7.500 |
| Trisodium Citrate | GM | 2.500 |
| Water for injection | ML | Q.S. to 100.0 |

### Example 16

The method for preparation of the lyophilized Sovateltide-based injectable formulation comprises of following steps: i) 1.500 gm of Trisodium Citrate Dihydrate is dissolved in 75 ml of water for injection in order to obtain a mixture, ii) 0.900 mg of Sovateltide is added in the mixture, followed by addition of 4.800 gm of Mannitol in order to obtain a solution, iii) the pH of the solution is checked, for making the volume of the solution, followed by filtering the solution and filling the filtered solution in vials, and iv) the plugs on the vials are half stoppered, followed by loading the vials in freeze dryer for lyophilization to obtain the lyophilized Sovateltide-based injectable formulation. Table 16 represents the ingredients along with the composition used in the preparation of lyophilized Sovateltide-based injectable formulation.

TABLE 16
Ingredients along with the composition used in the preparation of
lyophilized Sovateltide-based injectable formulation (Example 16)

| | Quantity used | | |
|---|---|---|---|
| Ingredient | (Batch size 30 vials) | Quantity per vial | |
| Sovateltide | 0.900 mg | 0.03 | mg |
| Mannitol | 4.800 gm | 160 | mg |
| Trisodium Citrate | 1.500 gm | 50 | mg |
| Water for injection | Q.S. to 75.0 ml | 2.5 | ml |

### Results of Different Trials:

Referring to Table 16, a tabular representation of the outcomes of various experiments, which were conducted to investigate certain parameters is illustrated.

TABLE 16
Result of Trial

| | Parameters | | | | | |
|---|---|---|---|---|---|---|
| | | Clarity & | | | | |
| Trial | | Color of | | | Parameter (Assay) | |
| Batch | | reconstituted | Osmolarity | Assay | At | At |
| No. | Description | solution | (mOsmol/Lit) | (2-8° C.) | (2-8° C.) | (25° C.) |
| Example 1 | Complies | Complies | 266 | 100.97% | 100.61 | 100.51% |
| Example 2 | Complies | Complies | Dœes not | 93.68% | — | 93.93% |
| | | | comply | | | |
| Example 3 | Complies | Complies | Dœes not | 90.85% | — | 86.19% |
| | | | comply | | | |
| Example 4 | Complies | Dœes not | Dœes not | 94.58% | — | 88.61% |
| | | comply | comply | | | |
| Example 5 | Complies | Dœes not | Dœes not | 98.43% | — | 84.81% |
| | | comply | comply | | | |
| Example 6 | Complies | Dœes not | Dœes not | 92.24% | — | 84.12% |
| | | comply | comply | | | |
| Example 7 | Complies | Dœes not | Dœes not | 104.88% | — | 100.22% |
| | | comply | comply | | | |
| Example 8 | Complies | Dœes not | Dœes not | 109.21% | — | 97.27% |
| | | comply | comply | | | |
| Example 9 | Complies | Complies | Dœes not | Not done | — | Not done |
| | | | comply | | | |
| Example 10 | Complies | Dœes not | Dœes not | Not done | — | Not done |
| | | comply | comply | | | |
| Example 11 | Complies | Complies | Dœes not | Placebo | — | Placebo |
| | | | comply | | | |
| Example 12 | Complies | Complies | Dœes not | Placebo | — | Placebo |
| | | | comply | | | |
| Example 13 | Complies | Complies | Dœes not | Not done | — | Not done |
| | | | comply | | | |
| Example 14 | Complies | Complies | 263 | Placebo | — | Placebo |
| Example 15 | Complies | Complies | 275 | Placebo | — | Placebo |

The results of Example 1 were analyzed in accordance with the in-house specifications for Description, Clarity, Osmolarity and Assay, and found to meet these standards after one month of storage at a temperature of 25° C. As a result of these findings, pilot batches were produced for further testing and investigation.

Referring to FIG. 1, a flow chart of a process of developing an injectable formulation involves several steps are illustrated. The process of manufacturing Sovateltide injection 30 μg is explained in the flow chart.

### In Process Specification:

Referring to Table 17, a tabular representation of an outline of specific parameters and requirements that must be met during the manufacturing of the formulation is illustrated. During the manufacturing process, various parameters are closely monitored to ensure that the final product meets the required standards. Among these, parameters are the description of the product, pH of the solution, assay, sterility, bacterial endotoxin test, and bioburden. After analysis, it was determined that the outcomes or results that were obtained fell within the predefined or specified range or limit.

TABLE 17
Outlines of specific parameters and requirements that
must be met during the manufacturing of the formulation

| | | |
|---|---|---|
| S.No. | Test | Specification |

Before Filtration

| | | |
|---|---|---|
| 1. | Description | Clear and Colorless solution |
| 2. | pH of solution | Between 7.5 to 8.5 |
| 3. | Bioburden | NMT 100 CFU/100 mL |

After Filtration

| | | |
|---|---|---|
| 1. | Description | Clear and Colorless solution |
| 2. | pH of solution | Between 7.5 to 8.5 |
| 3. | Assay (By | Not less than 90.0% w/w and not more |
| | HPLC) | than 110.0% w/w of the labelled amount |
| | | of Sovateltide |
| 4. | Sterility | The sample should be free from |
| | | microorganisms |
| 5. | Bacterial | Should not be more than 100 EU/mL |
| | endotoxin test | |
| 6. | Bioburden | Should not be more than 10 CFU/100 mL |

### Determination of Freeze-Drying Cycles:

The vials filled with the filtered solution are subjected to lyophilization by loading in freeze dryer. The process involves three main stages: i) freezing, ii) primary drying, and iii) secondary drying. i) The primary drying stage is particularly critical and involves the sublimation of ice from the frozen product. In this case, the freeze-drying cycle to make the formulation lyophilized has been selected as follows. Firstly, the freezing stage is set to −45° C. This extremely low temperature ensures that the substance is frozen quickly and effectively. This step is essential to prevent the formation of large ice crystals that could damage the product's structure and composition.

After the freezing stage, ii) the primary drying stage begins. The temperature is set to −20° C. for specific duration. During this time, the ice crystals in the substance start to sublimate, turning from a solid state to a gaseous state. This process removes most of the water content from the product. iii) The next stage of primary drying is set to 0° C. for specific duration. This slower process ensures that all the remaining water content is removed from the product. Finally, the temperature is set to +10° C. for specific duration in the last stage of primary drying. At this temperature, any residual ice in the product sublimates, and the drying process is complete. The final product is a lyophilized formulation that can be stored for extended periods at 2° C.-8° C. or 22° C. to 28° C.

Employment of freeze-drying yields multiple benefits, which can be outlined as follows:
a) The present invention pertains to an injectable drug product possessing favorable attributes of physical appearance and rapid redissolution. The incorporation of mannitol in suitable proportions in the formulation has enabled the development of a facile and uncomplicated prescription with excellent flexibility, leading to the production of Sovateltide for injection. The addition of mannitol has facilitated the freezing of the solution, and the resultant dried frozen products exhibit enhanced solubility and clarity upon re-dissolution. Furthermore, the impurity content is low, and the stability of the product can be efficiently monitored, ensuring that the quality is well-regulated.
b) The product exhibits long-term stability and superior quality. One aspect of this embodiment pertains to freeze-dried formulations of Sovateltide. Further, the lyophilized preparation is packaged within a vial or another container that is appropriate for pharmaceutical use.

Thus, it may be concluded that the products derived from freeze drying process hold a notably low moisture content with a limit under 7.0% and can be stored at cold temperatures or at room temperature. The products exhibited no impurity content and maintained their original color as lyophilized injectable powder/cake. Upon redissolving, the resulting solution retains its clarity and exhibits no significant change. This overall demonstrates a high level of product stability. Bacterial endotoxin test result complies with the regulatory requirement, performed by Gel clot technique Limulus Amebocyte Lysate (LAL) reagent.

### Finished Product Specification:

Referring to Table 18, a tabular representation outlines the various parameters that have been established to ensure the quality and consistency of the finished product. The lyophilized sovateltide injection underwent an analysis of specified parameters, including its description, identification, reconstitution time, and appearance after reconstitution. The results of the analysis revealed that all of these parameters meet the required specifications, indicating that the product has passed the necessary quality standards. The reconstituted solution was found to be within the specified pH range of 7.0-8.5, with a pH value of 8.32. The moisture content, determined using the Karl Fischer method, was 2.61% w/w, well within the allowed limit of not more than 7%. Bacterial endotoxins were found to comply with regulatory requirements, and the amount of particulate matter for particles ≥10 μm and ≥25 μm were 81 and 02 per container, respectively, which were well within the limit. Specified impurity was not detected while unidentified impurity and total impurity was 0.39% and 0.87%, respectively. The assay value was observed to be 95.65% with osmolarity of 275 mOsm/L. These results of the batch analysis confirm the success of the manufacturing process development. Lyophilized Sovateltide Injection, 30 μg should be stored between 2 to 8° C., protect from light.

TABLE 18
Outlines the various parameters that have been established to ensure the quality
and consistency of the finished product (i.e. disclosed in EXAMPLE 1)

| S. No. | Test | Specification |
|---|---|---|
| 1. | Description | White to off-white lyophilized cake. |
| 2. | Reconstitution Time | Content should dissolve within 1 minute. |
| 3. | Clarity of | The solid should dissolve completely and the |
| | reconstituted | solution should be visually clear. |
| | solution | |
| 4. | Identification | |
| | A. By HPLC | The retention time of Sovateltide peak in |
| | (By UV Detector) | chromatogram of the test sample should |
| | | correspond to that in the chromatogram of the |
| | | reference standards as obtained in assay method. |
| | B. By HPLC | The PDA spectrum of Sovateltide in test sample |
| | (By PDA Detector) | should correspond to that of the reference |
| | | standard as obtained in assay method. |
| 5. | Particulate Matter | Particle ≥10 μm should not exceed 6000 per container. |
| | | Particle ≥25 μm should not exceed 600 per container. |
| 6. | pH | Between 7.5 to 8.5. |
| 7. | Moisture Content | Not more than 7.0%. |
| | (% w/w) | |
| 8. | Assay (%) | Not less than 90.0 and not more than 110.0 of |
| | | labelled amount of Sovateltide. |
| 9. | Related | Specified Impurity |
| | Substances (%) | D-His-Sovateltide: NMT 1.0 |
| | | Unspecified Impurity: NMT 0.5 |
| | | Total Impurity: NMT 2.0 |
| 10. | Bacterial | Should not be more than 16666 EU/mg |
| | Endotoxin test | |
| 11. | Sterility Testing | Sample should be free from microorganisms |
| 12. | Osmolarity | Between 240 to 310. |
| | (mOsm/L) | |
| 13. | Uniformity of | Maximum acceptance value should be less than |
| | dosage unit (By | or equal to 15.0. |
| | Content | |
| | Uniformity | |

### Packaging Configuration:

The formulation in the vials was further packaged. The configurations of which are mentioned below:
Glass Vial—5 mL, USP type 1, Amber, tubular glass vial.
Rubber Stoper—13 mm Slotted Grey Bromo butyl “RFU” Rubber Stoppers.
Aluminum Seal—13 mm Aluminum Flip-Off Seal.

Referring to Table 19 and 20, a tabular representation of the specification and results of container and closure system are illustrated, respectively.

TABLE 19
The specifications for 13 mm Aluminum Flip-Off Tear-Off Seal

| Sr. No. | Description | Specification (mm) |
|---|---|---|
| 1 | Inner Diameter of Aluminum Seal | 13.00-13.60 |
| 2 | Outer Diameter of Aluminum Seal | 13.50-14.10 |
| 3 | Diameter of Plastic Disc | 14.50-15.00 |
| 4 | Height of Plastic Disc | 2.90 m-3.30 |
| 5 | Inner Height of Alum. Seal | 6.10-6.40 |
| 6 | Assembled Height | 7.62-8.38 |
| 7 | Thickness of Aluminium Cap | 0 18-0 20 |
| 8 | No. of Bridges | 06 Nos |
| 9 | Approved Drawing No | FO/VS/DE-13 |

As shown in Table 19, the packaging component Aluminum Flip-Off Tear-Off Seal (13 mm) was used with very precise measurements. The seal is designed to fit securely on a container with an inner diameter of 13.32 to 13.40 mm, while its outer diameter ranges from 13.78 to 13.90 mm. The plastic disc, which is an integral part of the seal and used to cover the top of the vial, has a diameter of 14.75 to 14.83 mm and a height of 3.02 to 3.10 mm. The inner height of the aluminum seal, which is the distance between the bottom of the seal and the top of the plastic disc, ranges between 6.25 mm to 6.32 mm. The assembled height of the seal, which is the distance between the bottom of the seal and the top of the plastic disc ranges between 7.81 mm to 8.02 mm. The thickness of the aluminum cap is 0.18 mm, which ensures that the seal is sturdy. Additionally, the seal complies with the approved drawing, ensuring that it meets all the necessary requirements.

TABLE 20
The specifications for 13 mm Grey Bromobutyl “RFU” Rubber stoppers

| S. No. | TESTS | LIMITS |
|---|---|---|
| 1. | Description | 13 mm slotted grey coloured Bromobutyl |
| | | “RFU” rubber stoppers |
| 2. | Physical Parameter | |
| | Disc Diameter | 12.50 mm to 12.80 mm |
| | Flanges Thickness (Collar) | 02.00 mm to 02.40 mm |
| | Maximum Height | 10.20 mm to 10.80 mm |
| | Diameter | 07.70 mm to 08.10 mm |
| 3. | Fitment with vial | Should be satisfactory |
| 4. | Identification by IR | IR spectra of sample should be concordant |
| | | with the standard spectra of |
| | | working/reference standard of the rubber |
| | | stopper |
| 5. | Total Ash | 41.40% to 50.60% |
| 6. | Appearance of solution | Solution A should not be more opalescent |
| | (Opalescence and Colour) | than opalescence standard OS3 and not |
| | | more intensely coloured than reference |
| | | solution BYS6. |
| 7. | Acidity or Alkalinity | Not more than 0.3 ml of 0.01M NaOH |
| | | should be required to produce blue colour |
| 8. | Light Absorption | Absorbance should not be more than 2.0 |
| | | in the range of 220 to 360 nm |
| 9. | Reducing substance | The difference between the titration |
| | | volumes should not be more than 7.0 ml |
| 10. | Heavy Metals | Not more than 20 ppm |
| 11. | Residue on Evaporation | Not more than 4.0 mg |
| 12. | Volatile sulphides | Black stains on paper should not be more |
| | | intense than that of a standard. |
| 13. | Fragmentation Test | Total number of fragments should not be |
| | | more than 15 |
| 14. | Sterilization test | The closures ‘prepared’ should not soften |
| | | or become tacky and there should be no |
| | | visual change in the closure |
| 15. | Liquid Particulate Test | Not more than 600 |
| 16. | | Not more than 60 |
| 17. | Sterility Test | No growth of micro-organism should |
| | | occur after 14 days of incubation. |
| 18. | Endotoxin Test | Not more than 0.25 EU/ml |

As shown in Table 20, that the 13 mm slotted grey colored Bromobutyl “RFU” rubber stoppers meet the requisite physical parameters. The diameter of the disc measures 12.68 mm, while the flanges thickness or collar measures 2.21 mm, and the maximum height is 10.47 mm. The diameter of the rubber stopper is 7.87 mm, and it demonstrates an optimal fitment with the vial. The IR spectra of the sample are concordant with the reference standard, enabling accurate identification of the rubber stopper. The appearance of solution A is consistent with acceptable opalescence and colour standards, with the solution demonstrating no more opalescence than opalescence standard OS3 and not more intensely coloured than reference solution BYS6. The rubber stopper manifests no significant acidity or alkalinity (0.19 mL) and is within the specified range, while light absorption (0.0028) and reducing substances (1.9 mL) are within the acceptable range. The heavy metal concentration is less than 20 ppm, and residue on evaporation is minimal i.e., 2.1 mg, indicating adherence to the desired parameters. The rubber stopper passed the sterilization test, demonstrating that it does not soften or become tacky, and there is no visual change in the closure. Further, the liquid particulate test results show that the number of particles that measure ≥10 μm is 17, while the number of particles >25 μm is 1, meeting the set specifications. The sterility test demonstrated that there was no growth observed, and the endotoxin test showed that the endotoxin concentration was less than 0.25 EU/ml, signifying compliance with the desired quality standards. Therefore, the 13 mm slotted grey coloured Bromobutyl “RFU” rubber stoppers have successfully met the requisite physical and chemical specifications, demonstrating their suitability for use.

Referring to Table 21, a tabular representation of a specification for Amber color vial is illustrated. The results present an exhaustive investigation of the physical parameters of a 5 ml amber color vial. The height of the vial was measured and was found to range from 48.00 mm to 48.73 mm, with an average height of 48.36 mm. Additionally, the rim height of the vial was observed to vary from 3.60 mm to 3.91 mm, with an average height of 3.75 mm. The diameter of the vial's body was found to range from 16.16 mm to 16.37 mm, with an average diameter of 16.27 mm. Furthermore, the mouth outer diameter of the vial was determined to vary from 12.94 mm to 13.02 mm, with an average diameter of 12.98 mm. Additionally, the mouth inner diameter of the vial was found to vary from 6.93 mm to 7.18 mm, with an average diameter of 7.05 mm. Furthermore, it was established that the T1 body wall thickness and T2 bottom wall thickness of the vial were both in compliance with the prescribed standards. The meticulous data collected from the analysis conclusively indicates that the 5 ml amber color vial is well within the specified range of parameters and meets the required standards. The vial has undergone rigorous visual testing and has been found to be in compliance with industry standards. The vial is made from borosilicate amber USP type glass that meets the requirements for construction. Furthermore, the product is free from dust, dirt, or any foreign particles that may affect its quality or function. In addition to this, the vial has also been evaluated for any chipping or bubbles that may compromise its integrity. As per the results, it complies with the set standards, indicating that it is free from any visible imperfections (Table 21). Furthermore, vial has been subjected to various tests including glass grain test, surface glass test, arsenic test and the results indicate compliance with the standards (Table 21).

TABLE 21
Specification for Amber color vial

| | |
|---|---|
| Parameters | Specification (mm) |

Analytical Report

| | |
|---|---|
| Total height (H1) | 48.00-49.00 |
| Rim height (H2) | 3.50-4.10 |
| Body diameter (D1) | 16.00-16.50 |
| Mouth outer diameter (D2) | 12.70-13.20 |
| Mouth inner diameter (D3) | 6.80-7.20 |
| Body wall thickness (T1) | NLT-0.80 |
| Bottom wall thickness (T2) | NLT-0.70 |

Visual Analysis

| | |
|---|---|
| Material of construction | Borosilicate amber USP type glass |
| Dust dirt or foreign particles | Absent |
| Chipping and bubbles | Absent |

Glass Quality

| | |
|---|---|
| Glass grain test | Maximum volume of 0.02M HCl/g. of test glass |
| | should be not more than 0.1 mL. |
| Surface glass test | Maximum volume of 0.01M HCI. per 100 ml. of test |
| | liquid should be not more than 1.00 ml. |
| Arsenic test | Not more than 0.1 ppm |

### Stability Studies:

The stability of drug product—Sovateltide for injection 30 μg stored in USP type I glass vial conducted at 25° C.±2° C. and 60% RH±5% RH for not less than 12 months, wherein each vial contains 30 μg of Sovateltide. Initially the pH of the lyophilized injectable was 7.91 and after 12 months the pH was 7.67 and which is within the specified limit. The Reconstitution time, Clarity of reconstituted solution, moisture content, sterility, Bacterial Endotoxins limit, Particulate matter, Related Substances (%), all the parameters meet the regulatory requirements and are within the specified limit. Initially, the percent assay of lyophilized Sovateltide injection was 100.41% and after storage at 25° C.±2° C. and 60% RH±5% RH for not less than 12 months, the assay value was 98.81%. The results confirm that the formulation remains stable for not less than 12 months at a storage temperature of 25° C.±2° C.

Similarly, the developed pharmaceutical lyophilized injectable formulation of Sovateltide stored at a temperature ranging between 2° C.-8° C., is stable not less than 36 months of storage and the results complies with specified reconstitution time, clarity of reconstituted solution, moisture content, sterility, bacterial endotoxins limit, particulate matter, related substances (%). Initially, the pH of the lyophilized injectable stored at 5° C.±3° C. was 8.18 and after 36 months the pH was 7.77. Initially, percent assay of the lyophilized Sovateltide injection stored at 5±3° C. was 106.00% and after storage of formulation for not less than 36 months, the assay value was 95.58%. The results are in favor that the formulation remains stable for not less than 36 months at storage temperature of 5±3° C.

### Force Degradation Study:

Referring to Table 22, a tabular representation of a Force Degradation Study Sovateltide Injection is illustrated. Different conditions for forced degradation (chemical degradation) were set comprising 1 mL-5 N HCl/3 Hours RT; 1 mL-5 N NaOH/3 Hours RT; 1 mL-30% H₂O₂/3 Hours RT; for acid degradation, base degradation; and peroxide degradation, respectively. Different conditions for forced degradation (physical degradation) were set comprising 3 Hours/105° C.; 95% RH/24 Hours for Thermal Degradation and Humidity Degradation, respectively. Different conditions for forced degradation were set to observe photolytic degradation and the samples covered with Aluminum foil (Dark Condition); Final Packaging; Primary Packaging (Labelled); Primary Packaging (Unlabeled) and 1.2 million Lux hours & 200 Watt-Hour/m² (Open Condition). According to the findings, Lyophilized Sovateltide injection 30 μg demonstrated sensitivity to acid, base, peroxide, and photolytic degradation. The results of chromatography and peak purity analysis of the degraded samples revealed that the analyte peaks were uniform, with no co-eluting peaks from the degradation process. Sovateltide peak purity was maintained in all chromatograms under various stress conditions.

TABLE 22
Force Degradation Study Sovateltide Injection

| | | | | | | | | |
|---|---|---|---|---|---|---|---|---|
| | | | | | Single | | | |
| | | | | | Maximum | Total | | |
| | | Peak Purity | Peak Purity | Specified | Unknown | Unknown | Total | |
| | | Index of | Index of | impurity | Impurity | Impurities | Impurity | % |
| | | Sovateltide | Sovateltide | from | from | from | (Method-1 + | Degradation |
| Degradation | Degradation | from | from | Method-2 | Method-1 | Method-1 | Method-2) | (w.r.t. |
| Mode | Condition | Method-1 | Method-2 | (%) | (%) | (%) | (%) | Control) |

Chemical Degradation

| | | | | | | | | |
|---|---|---|---|---|---|---|---|---|
| Control | No | 0.999824 | 1.000000 | ND | 0.190 | 0.336 | 0.336 | — |
| | Treatment | | | | | | | |
| Acid | 1 mL-5N | 0.999992 | 0.999049 | ND | 2.937 | 3.195 | 3.195 | 2.86 |
| Degradation | HCl/3 | | | | | | | |
| | Hours RT | | | | | | | |
| Base | 1 mL-5N | 1.000000 | 1.000000 | ND | 2.239 | 4.639 | 4.639 | 4.30 |
| Degradation | NaOH/3 | | | | | | | |
| | Hours RT | | | | | | | |
| Peroxide | 1 mL-30% | 1.000000 | 1.000000 | ND | 1.840 | 6.830 | 6.830 | 6.49 |
| Degradation | H₂O₂/3 | | | | | | | |
| | Hours RT | | | | | | | |

Physical Degradation

| | | | | | | | | |
|---|---|---|---|---|---|---|---|---|
| Thermal | 3 hours/ | 0.989942 | 1.000000 | ND | 1.357 | 5.199 | 5.199 | 4.86 |
| Degradation | 105° C. | | | | | | | |
| Humidity | 95% RH/24 | 0.997178 | 1.000000 | ND | 0.280 | 0.740 | 0.740 | 0.40 |
| Degradation | Hours | | | | | | | |

Photolytic Degradation

| | | | | | | | | |
|---|---|---|---|---|---|---|---|---|
| Photolytic | Covered | 0.989942 | 1.000000 | ND | 1.485 | 1.964 | 1.964 | 1.63 |
| Degradation | with | | | | | | | |
| | Aluminium | | | | | | | |
| | foil (Dark | | | | | | | |
| | Condition) | | | | | | | |
| | Final | 0.997178 | 0.999609 | ND | 0.249 | 0.846 | 0.846 | 0.51 |
| | Packaging | | | | | | | |
| | Primary | 1.000000 | 1.000000 | ND | 1.924 | 5.604 | 5.604 | 5.27 |
| | Packaging | | | | | | | |
| | (Labelled) | | | | | | | |
| | Primary | 1.000000 | 1.000000 | ND | 1.924 | 4.567 | 4.567 | 4.23 |
| | Packaging | | | | | | | |
| | (Unlabeled) | | | | | | | |
| | 1.2 million | 0.999845 | 0.999998 | ND | 0.786 | 2.940 | 2.940 | 2.60 |
| | Lux hours & | | | | | | | |
| | 200 Watt- | | | | | | | |
| | Hour/m2 | | | | | | | |
| | (Open | | | | | | | |
| | Condition) | | | | | | | |

### Method Validation for Assay of Sovateltide Injection:

Chromatographic Parameters for method validation of Assay of Sovateltide Injection: An analytical system comprising an Xtimate C18 (250×4.6 mm), 5 μm or Chromcore 120 C18 (250×4.6 mm), 5 μm or an analytical column that was equivalent was utilized for the experiment. The flow rate was maintained at 0.8 mL/min, while the injection volume was 80 μL. The detection wavelength was set at 215 nm, and the total run time was 30 minutes. The autosampler temperature was 5° C. and the column oven temperature was 40° C. The retention time was determined to be 13±2 minutes.

### Mobile Phase for Assay:

Mobile Phase-A, (10 mM KH₂PO₄ & 10 mM NaCl, pH− 7.5)
Mobile Phase-B (Methanol)

Referring to Table 23, a tabular representation of a Method Validation Summary of Assay of Sovateltide Injection is illustrated. The method validation for the assay of Sovateltide in Sovateltide for injection 30 μg was done by HPLC. After the evaluation of data, the method was found precise, linear, accurate, robust, rugged, and specific. As the results of all the validation parameters were within the acceptance criteria, it is concluded that the analytical method is suitable for the determination of Sovateltide by HPLC. Based on solution stability study, the Standard solution of Sovateltide was stable for up to 24 Hours at 5° C., sample solution of Sovateltide injection was stable for up to 26 Hours at 5° C. and sample solution of In-process bulk solution was found stable for up to 25 Hours at 5° C. (Table 23).

TABLE 23
Validation Summary of Assay of Sovateltide Injection

| S. | Validation | | |
|---|---|---|---|
| No. | Parameter | Results | Acceptance Criteria |
| 1. | Specificity | No interference is observed at | In blank solution |
| | A. Interference | retention time of Sovateltide peak | chromatogram, there should |
| | Studies | in blank & placebo solutions. | not be any peak interference |
| | | Sovateltide peak was found to be | at the retention time of |
| | | homogeneous and peak purity is | Sovateltide peak. |
| | | passed in standard and sample | In the placebo solution |
| | | solution. | chromatogram, there should |
| | | | not be any peak interference |
| | | | at the retention time of the |
| | | | Sovateltide peak. |
| | | | In the sample solution |
| | | | chromatogram, peak purity |
| | | | of the Sovateltide peak |
| | | | should pass. |
| | B. Force | Based on the observed results, | All unknown peaks should |
| | Degradation | Lyophilized Sovateltide for | be resolved from |
| | Studies | injection was found sensitive | Sovateltide Peak. |
| | | towards acid, base & peroxide | The peak purity of the |
| | | and photolytic degradation. | Sovateltide peak in all |
| | | The chromatograms and peak | stressed samples should |
| | | purity values of degradation | pass. |
| | | sample solutions indicate that the | Degradation should not be |
| | | analyte peaks are homogeneous | more than 30% in any |
| | | and there are no co-eluting peaks | condition. |
| | | from degradation. | |
| | | Sovateltide peak purity in each | |
| | | stress conditions chromatograms | |
| | | were passed. | |
| 2. | Precision | The percentage relative standard | The percentage relative |
| | A. System | deviation (% RSD) of area counts of | standard deviation (% RSD) of |
| | precision | Sovateltide peak from six replicate | area counts of Sovateltide peak |
| | | injections of standard solution were | from six replicate injections of |
| | | found 1.61% which is within | standard solution should not be |
| | | acceptance criteria. | more than 10.0%. |
| | B. Method | The % RSD for unspecified | Each sample should meet |
| | Precision | impurities above LOQ (0.1%) & | the specification. |
| | | total unspecified impurities of six | % RSD results for each |
| | | sample preparations was within the | individual impurity which |
| | | acceptance criteria. The study reveals | are above limit of |
| | | that the test results obtained by this | quantification (0.1%) from |
| | | method are repeatable and the | six sample preparations |
| | | method was found to be precise. | should not be more than |
| | | | 10.0%. |
| | C. Intermediate | The % RSD of each unspecified | Each sample should meet |
| | Precision & | impurity above LOQ (0.1%) & | the specification. |
| | Ruggedness | total unspecified impurity from | % RSD results for each |
| | | six samples were observed within | individual impurity which |
| | | the acceptance criteria. | are above the limit of |
| | | The overall % RSD of each | quantification (0.1%) from |
| | | unspecified impurity & total | six sample preparations |
| | | unspecified impurity of replicate | should not be more than |
| | | test preparations (12 results) of | 10.0%. |
| | | method precision and | Overall % RSD of results of |
| | | intermediate precision was | replicate test preparations |
| | | observed within the acceptance | (12 results) of method |
| | | criteria. | precision and intermediate |
| | | The study concludes that the | precision should not be |
| | | method was found to be rugged | more than 10.0% for each |
| | | and precise against variabilities | individual impurity which |
| | | like two different HPLC | are above limit of |
| | | instruments, analysts, columns, | quantification (0.1%). |
| | | days. | |
| 3. | A. Limit of | The limit of detection for | % RSD of area counts for |
| | Detection | Sovateltide was observed at 0.03 | Sovateltide shall not be more |
| | | % with respect to sample | than 33.0% for limit of |
| | | concentration. | detection. |
| | | % RSD at limit of detection was | |
| | | observed 6.62% for Sovateltide, | |
| | | which is within the acceptance | |
| | | criteria. | |
| | B. Limit of | The limit of quantitation for | % RSD of area counts for |
| | Quantitation | Sovateltide was observed at 0.101% | Sovateltide shall not be more |
| | | with respect to sample concentration. | than 10.0% for limit of |
| | | % RSD at limit of quantitation | quantitation. |
| | | was observed 3.01% for | |
| | | Sovateltide, which is within the | |
| | | acceptance criteria. | |
| 4. | Linearity | The correlation coefficient was | Correlation coefficient |
| | | observed 0.9998 for Sovateltide, | should not be less than 0.98. |
| | | which was meeting the | For detector linearity the % |
| | | acceptance criteria. | Y-intercept at 100% |
| | | Detector linearity for Sovateltide | response level should be |
| | | the % Y-intercept at 100% | within ±2.0. |
| | | response level was observed at | |
| | | 0.0204, which was meeting the | Individual % recovery and |
| | | acceptance criteria. | mean % recovery at LOQ |
| 5. | Accuracy | The method was found accurate, | level should be in between |
| | | and all results were within | 80.0% and 120.0%. |
| | | acceptance criteria. | Individual % recovery and |
| | | Individual and overall % | mean % recovery at other |
| | | recovery, mean % recovery & % | levels should be in between |
| | | RSD results were found meeting | 90.0% and 110.0%. |
| | | the acceptance criteria. | % RSD should not be more |
| | | Overall % RSD was found 5.18%. | than 10.0% at each level. |
| | | | Overall % RSD should not |
| | | | be more than 10.0%. |
| 6. | Range | From the data of precision, | Acceptance criteria mentioned |
| | | linearity, and accuracy, it is | under the validation parameters |
| | | observed that the method has a | of linearity, accuracy and |
| | | range of 0.018 μg/mL to 0.440 | precision should pass. |
| | | μg/mL for standard of Sovateltide. | |
| | | All acceptance criteria mentioned | |
| | | under the validation parameters of | |
| | | linearity, accuracy and precision | |
| | | were found within acceptance | |
| | | criteria. | |
| 7. | Stability of | From the results it is concluded that | % Cumulative RSD for |
| | Analytical | standard is stable in solution for up | standard solution should not |
| | Solution | to 60 Hours and control unspiked | be more than 10.0%. |
| | | sample is stable in solution for up to | % Impurity above reporting |
| | | 12 Hours at 5° C. | threshold (0.1%) and |
| | | | below 0.5% should not |
| | | | differ from the initial value |
| | | | by more than ±30.0%. |
| | | | % Impurity above 0.5% |
| | | | should not differ from the |
| | | | initial value by more than ±10.0%. |
| | | | No new peak should appear |
| | | | from the initial sample. |
| 8. | Robustness | The method remains unaffected | Should comply with the |
| | | due to all deliberate changes to | system suitability parameters |
| | | the analytical method. Hence, the | as per the test method. |
| | | method is robust for the intended | RT should be comparable. |
| | | use. | |
| 9. | System | Column efficiency (USP Plate | The column efficiency |
| | Suitability | Count) for Sovateltide peak from | (USP plate count) |
| | | first injection of standard solution | determined from the |
| | | from each parameter and each | Sovateltide peak should not |
| | | robustness condition were found | be less than 2000 |
| | | within the acceptance criteria. | theoretical plates from first |
| | | The % relative standard deviation | injection of standard |
| | | of area counts of Sovateltide peak | solution. |
| | | from three replicate injections of | The percentage relative |
| | | standard solution from each | standard deviation (% RSD) |
| | | parameter and each robustness | of area counts of |
| | | condition were found within the | Sovateltide peak from three |
| | | acceptance criteria. | replicate injections of |
| | | | standard solution should not |
| | | | be more than 10.0%. |

### Related Substances Method Validation Summary:

Chromatographic Parameters for method validation of Related Substance of Sovateltide Injection (Method 1): Method-1 utilizes chromatographic conditions to separate and quantify a specific compound of interest. The analytical column used is either the Xtimate C18 Column or the ChromCore 120 C18 Column, both with dimensions of 4.6×250 mm and 5 m. A Ghost Buster Column with dimensions of 4.6×50 mm is used to detect any unexpected peaks, which are usually called ghost peaks that may interfere with the results. The flow rate is set to 0.5 mL/min, with an injection volume of 90 μL. The detection wavelength is 215 nm, and the auto sampler temperature is maintained at 5° C. The column oven temperature is set to 50° C., and the run time for the method is 120 min. The retention time for the compound of interest is approximately 66.0 min under these chromatographic conditions. These parameters ensure the separation and accurate quantification of the compound in the sample.

### Mobile Phase for Un-Specified Impurities (Method 1):

Mobile Phase A (KH2PO4+ Sodium Chloride in HPLC water).
Mobile Phase-B (Methanol).

Referring to Table 24, a tabular representation of a Method Validation Summary of Related Substance in Sovateltide Injection (Method 1) is illustrated. After evaluation of data, the method was found precise, linear, accurate, robust, rugged and specific. As the results of all the validation parameters were within the acceptance criteria, it is concluded that the analytical method is suitable for the determination of organic impurities (unspecified) in Lyophilized Sovateltide for Injection by HPLC. Based on solution stability study, standard solution was stable up to 60 Hours and sample solution was stable up to 12 Hours at 5° C. (Table 24).

TABLE 24
Method Validation Summary of Related Substance in Sovateltide Injection (Method 1)

| S. | Validation | | |
|---|---|---|---|
| No. | Parameter | Results | Acceptance Criteria |
| 1. | Specificity | No interference is observed at | In blank solution |
| | A. Interference | retention time of Sovateltide | chromatogram, there should |
| | Studies | peak in blank & placebo | not be any peak interference |
| | | solutions. | at the retention time of the |
| | | Sovateltide peak was found to | Sovateltide peak. |
| | | be homogeneous and peak | In the placebo solution |
| | | purity is passed in standard and | chromatogram, there should |
| | | sample solution. | not be any peak interference |
| | | | at the retention time of the |
| | | | Sovateltide peak. |
| | | | In the sample solution |
| | | | chromatogram, peak purity |
| | | | of the Sovateltide peak |
| | | | should pass. |
| | B. Force | Based on the observed results, | All unknown peaks should |
| | Degradation | Lyophilized Sovateltide for | be resolved from Sovateltide |
| | Studies | injection was found sensitive | Peak. |
| | | towards acid, base & peroxide | The peak purity of |
| | | and photolytic degradation. | Sovateltide peak in all |
| | | The chromatograms and peak | stressed samples should |
| | | purity values of degradation | pass. |
| | | sample solutions indicate that | Degradation should not be |
| | | the analyte peaks are | more than 30% in any |
| | | homogeneous and there are no | condition. |
| | | co-eluting peaks from | |
| | | degradation. | |
| | | Sovateltide peak purity in each | |
| | | stress conditions | |
| | | chromatograms were passed. | |
| 2. | Precision | The percentage relative standard | The percentage relative standard |
| | D. System | deviation (% RSD) of area counts | deviation (% RSD) of area |
| | precision | of Sovateltide peak from six | counts of Sovateltide peak from |
| | | replicate injections of standard | six replicate injections of |
| | | solution were found 1.61% which | standard solution should not be |
| | | is with in acceptance criteria. | more than 10.0%. |
| | E. Method | The % RSD for unspecified | Each sample should meet the |
| | Precision | impurities above LOQ (0.1%) & | specification. |
| | | total unspecified impurities of six | % RSD results for each |
| | | sample preparations was within | individual impurity which |
| | | the acceptance criteria. The study | are above limit of |
| | | reveals that the test results | quantification (0.1%) from |
| | | obtained by this method are | six sample preparations |
| | | repeatable and the method was | should not be more than 10.0%. |
| | | found to be precise. | |
| | F. Intermediate | The % RSD of each | Each sample should meet the |
| | Precision & | unspecified impurity above | specification. |
| | Ruggedness | LOQ (0.1%) & total | % RSD results for each |
| | | unspecified impurity from six | individual impurity which |
| | | samples were observed within | are above limit of |
| | | the acceptance criteria. | quantification (0.1%) from |
| | | The overall % RSD of each | six sample preparations |
| | | unspecified impurity & total | should not be more than 10.0%. |
| | | unspecified impurity of | Overall % RSD of results of |
| | | replicate test preparations (12 | replicate test preparations |
| | | results) of method precision | (12 results) of method |
| | | and intermediate precision was | precision and intermediate |
| | | observed within the acceptance | precision should not be more |
| | | criteria. | than 10.0% for each |
| | | The study concludes that the | individual impurity which |
| | | method was found to be | are above limit of |
| | | rugged and precise against | quantification (0.1%). |
| | | variabilities like two different | |
| | | HPLC instruments, analysts, | |
| | | columns, days. | |
| 3. | A. Limit of | Limit of detection for | % RSD of area counts for |
| | Detection | Sovateltide was observed at | Sovateltide shall not be more |
| | | 0.03% with respect to sample | than 33.0% for limit of |
| | | concentration. | detection. |
| | | % RSD at limit of detection | |
| | | was observed 6.62% for | |
| | | Sovateltide, which is within | |
| | | the acceptance criteria. | |
| | C. Limit of | Limit of quantitation for | % RSD of area counts for |
| | Quantitation | Sovateltide was observed at | Sovateltide shall not be more |
| | | 0.101% with respect to sample | than 10.0% for limit of |
| | | concentration. | quantitation. |
| | | % RSD at limit of quantitation | |
| | | was observed 3.01% for | |
| | | Sovateltide, which is within | |
| | | the acceptance criteria. | |
| 4. | Linearity | Correlation coefficient was | Correlation coefficient |
| | | observed 0.9998 for | should not be less than 0.98. |
| | | Sovateltide, which was | For detector linearity the % |
| | | meeting the acceptance | Y-intercept at 100% |
| | | criteria. | response level should be |
| | | Detector linearity for | within ±2.0. |
| | | Sovateltide the % Y-intercept | |
| | | at 100% response level was | |
| | | observed at 0.0204, which was | |
| | | meeting the acceptance | |
| | | criteria. | |
| 5. | Accuracy | The method was found | Individual % recovery and |
| | | accurate, and all results were | mean % recovery at LOQ |
| | | within acceptance criteria. | level should be in between |
| | | Individual and overall % | 80.0% and 120.0%. |
| | | recovery, mean % recovery & | Individual % recovery and |
| | | % RSD results were found | mean % recovery at other |
| | | meeting the acceptance criteria. | levels should be in between |
| | | Overall % RSD was found 5.18% | 90.0% and 110.0%. |
| | | | % RSD should not be more |
| | | | than 10.0% at each level. |
| | | | Overall % RSD should not |
| | | | be more than 10.0%. |
| 6. | Range | From the data of precision, | Acceptance criteria mentioned |
| | | linearity, and accuracy, it is | under the validation parameters |
| | | observed that the method has a | of linearity, accuracy and |
| | | range of 0.018 μg/mL to 0.440 | precision should pass. |
| | | μg/mL for standard of Sovateltide. | |
| | | All acceptance criteria | |
| | | mentioned under the validation | |
| | | parameters of linearity, | |
| | | accuracy and precision were | |
| | | found within acceptance criteria. | |
| 7. | Stability of | From results it is concluded that | % Cumulative RSD for |
| | Analytical | standard is stable in solution for | standard solution should not |
| | Solution | up to 60 hrs. and control unspiked | more than 10.0%. |
| | | sample is stable in solution for up | % Impurity above reporting |
| | | to 12 Hours at 5° C. | threshold (0.1%) and below |
| | | | 0.5% should not differ from initial |
| | | | value by more than ±30.0%. |
| | | | % Impurity above 0.5% |
| | | | should not differ from initial |
| | | | value by more than ±10.0%. |
| | | | No new peak should appear |
| | | | from the initial sample. |
| 8. | Robustness | The method remains unaffected | Should comply with the |
| | | due to all deliberate changes to | system suitability parameters |
| | | the analytical method. Hence, | as per the test method. |
| | | the method is robust for the | RT should be comparable. |
| | | intended use. | |
| 9. | System | Column efficiency (USP Plate | The column efficiency (USP |
| | Suitability | Count) for Sovateltide peak | plate count) determined from |
| | | from first injection of standard | Sovateltide peak should not |
| | | solution from each parameter | be less than 2000 theoretical |
| | | and each robustness condition | plates from first injection of |
| | | were found within the | standard solution. |
| | | acceptance criteria. | The percentage relative |
| | | The % relative standard | standard deviation (% RSD) |
| | | deviation of area counts of | of area counts of Sovateltide |
| | | Sovateltide peak from three | peak from three replicate |
| | | replicate injections of standard | injections of standard |
| | | solution from each parameter | solution should not be more |
| | | and each robustness condition | than 10.0%. |
| | | were found within the | |
| | | acceptance criteria. | |

Chromatographic Parameters for method validation of Related Substance of Sovateltide Injection (Method 2): The given information describes the conditions and parameters used in an analytical column for a chromatographic analysis. The Ultisil XB-C30 Column or ChromCore C30 Column with dimensions of 4.6×250 mm and a particle size of 5 m is used as the analytical column. The Ghost-Buster Column with dimensions of 4.6×50 mm is used for removing. The flow rate is maintained at 0.5 mL/min, and an injection volume of 80 μL is used. The detection wavelength is set to UV at 215 nm, and the autosampler temperature is maintained at 5° C. while the column oven temperature is set at 50° C. The standard run time is 30 min, while it is 80 min for sample solutions. The retention time for the analysis is between 6 to 12 minutes, and the relative retention time is approximately 0.2 with respect to Sovateltide. These parameters are critical to obtaining accurate and reproducible results in a chromatographic analysis.

### Mobile Phase for Specified Impurities (Method 2): Mobile Phase A (0.05 M 1-Octane Sulfonic Acid Sodium Salt, pH 7.5):

Mobile Phase-B (Methanol).

Referring to Table 25, a tabular representation of a Validation Summary of Related Substance in Sovateltide Injection (Method 2) is illustrated. After evaluation of data, the method was found precise, linear, accurate, robust, rugged and specific. As the results of all the validation parameters were within the acceptance criteria, it is concluded that the analytical method is suitable for the determination of organic impurities (Specified—D-His-Sovateltide) in Lyophilized Sovateltide for Injection by HPLC. Based on solution stability study, standard solution was stable up to 48 Hours, spiked sample solution was stable up to 40 hrs and control sample solution was stable up to 39 Hours at 5° C. (Table 25).

TABLE 25
Validation Summary of Related Substance in Sovateltide Injection (Method 2)

| S. | Validation | | |
|---|---|---|---|
| No. | parameter | Results | Acceptance Criteria |
| 1. | Specificity | No interference is observed | In blank solution chromatogram, there |
| | A. Interference | at retention time of D-His | should not be any peak interference at |
| | Studies | peak. | the retention time of D-His & Sovateltide |
| | | D-His peak was found to be | peak. |
| | | homogeneous and peak | In Placebo solution chromatogram, there |
| | | purity is passed in standard | should not be any peak interference at |
| | | & spike sample solution. | the retention time of D-His & Sovateltide |
| | | Sovateltide peak was found | peak. |
| | | to be homogeneous and peak | In Main analyte (Sovateltide) solution |
| | | purity is passed in Main | chromatogram, there should not be any |
| | | analyte solution & sample | peak interference at the retention time of |
| | | and spike sample solution. | D-His peak. |
| | | | In Sample solution chromatogram, peak |
| | | | purity of D-His peak & Sovateltide peak |
| | | | should pass. |
| | | | In Spike sample solution chromatogram, |
| | | | peak purity of D-His peak & Sovateltide |
| | | | peak should pass. |
| | Force | Sovateltide peak purity in | No interference should be observed from |
| | Degradation | each stress conditions | placebo or diluent at retention time of D- |
| | Studies | chromatograms were passed. | His Peak. |
| | | The study concludes that the | Peak purity of Sovateltide peak in all |
| | | related substances method is | stressed samples should pass. |
| | | specific. | Peak purity of D-His peak in all stressed |
| | | All peaks are well resolved | samples should pass. |
| | | from Sovateltide peak. | Degradation should not be more than 30% |
| | | | in any condition. |
| 2. | Precision | The percentage relative | The percentage relative standard deviation |
| | G. System | standard deviation (% RSD) of | (% RSD) of area counts of D-His peak from |
| | precision | area counts of D-His peak from | six replicate injections of standard solution |
| | | six replicate injections of | should not be more than 5.0%. |
| | | standard solution were found | |
| | | 1.44% which is with in | |
| | | acceptance criteria. | |
| | H. Method | In Unspiked (Control) | Each sample should meet the |
| | Precision | sample no peak was found at | specification. |
| | | the RT of D-His. | % RSD of results of six sample |
| | | The % RSD of D-His of six | preparations should not be more than |
| | | spike sample preparations | 10.0%. |
| | | was observed 0.30%, which | |
| | | is within the acceptance | |
| | | criteria. | |
| | I. Intermediate | The % RSD of D-His (%) | Each sample should meet the specification. |
| | Precision | from six samples was | % RSD results for D-His from six spike |
| | | observed 6.17%, which is | sample preparations should not be more |
| | | within the acceptance | than 10.0%. |
| | | criteria. Data indicates that | Overall % RSD of results of replicate |
| | | the method has an acceptable | test preparations (12 results) of method |
| | | level of precision. | precision and intermediate precision |
| | | The overall % RSD of D-His | should not be more than 10.0% for D- |
| | | of replicate test preparations | His. |
| | | (12 results) of method | |
| | | precision and intermediate | |
| | | precision was observed 4.17%, | |
| | | which is meeting the | |
| | | acceptance criteria. | |
| 3. | A. Limit of | Limit of detection for D-His | % RSD of area counts for specified |
| | Detection | was observed at 0.06% with | impurity (D-His) shall not be more than |
| | | respect to sample | 33.0% for limit of detection. |
| | | concentration. | |
| | | % RSD at limit of detection | |
| | | were observed 9.40 for D- | |
| | | His, which is within the | |
| | | acceptance criteria. | |
| | D. Limit of | Limit of quantitation for D- | % RSD of area counts for specified |
| | Quantitation | His was observed at 0.18% | impurity (D-His) shall not be more than |
| | | with respect to sample | 10.0% for limit of quantitation. |
| | | concentration. | |
| | | % RSD at limit of | |
| | | quantitation were observed | |
| | | 5.75% for D-His, which is | |
| | | within the acceptance | |
| | | criteria. | |
| 4. | Linearity | Correlation coefficient was | Correlation coefficient should not be less |
| | | observed 0.9996 for D-His, | than 0.98. |
| | | which was meeting the | For detector linearity the % Y-intercept at |
| | | acceptance criteria. | 100% response level should be within ±2.0. |
| | | Detector linearity for | |
| | | Sovateltide the % Y- | |
| | | intercept at 100% response | |
| | | level was observed at 0.0401 | |
| | | and for D-His which was | |
| | | meeting the acceptance | |
| | | criteria. | |
| 5. | Accuracy | The method was found | Individual % recovery and mean % |
| | | accurate, and all results were | recovery at LOQ level should be in |
| | | within acceptance criteria. | between 80.0% and 120.0%. |
| | | Individual and overall % | Individual % recovery and mean % |
| | | recovery, mean % recovery | recovery at other levels should be in |
| | | & % RSD results were found | between 90.0% and 110.0%. |
| | | meeting the acceptance | % RSD should not be more than 10.0% |
| | | criteria. | at each level. |
| | | Overall % RSD for D-His | Overall % RSD should not be more than |
| | | was observed 8.18. | 10.0%. |
| 6. | Range | From the data of precision, | Acceptance criteria mentioned under the |
| | | linearity, and accuracy, it is | validation parameters of linearity, |
| | | observed that the method has | accuracy and precision should pass. |
| | | a range d 0.036 μg/mL to | |
| | | 0.401 μg/mL for D-His. | |
| | | All acceptance criteria | |
| | | mentioned under the | |
| | | validation parameters of | |
| | | linearity, accuracy and | |
| | | precision were found within | |
| | | acceptance criteria. | |
| 7. | Stability of | From results it is concluded that | % Cumulative RSD for standard solution |
| | Analytical | standard is stable in solution for | should not be more than 5.0%. |
| | Solution | up to 48 Hours, spiked sample | % Impurity above 0.5% should not differ |
| | | is stable in solution for up to 40 | from initial value by more than ±10.0%. |
| | | Hours and control unspiked | |
| | | sample is stable in solution for | |
| | | up to 39 Hours at 5° C. | |
| 8. | Robustness | The method remains unaffected | Should comply with the system |
| | | due to all deliberate changes to | suitability parameters as per the test |
| | | the analytical method. Hence, | method. |
| | | the method is robust for the | RT & RRT should be comparable. |
| | | intended use. | |
| 9. | System | The tailing factor for D-His | The tailing factor determined from D-His |
| | Suitability | peak from first injection of | peak should not more than 3.0 from first |
| | | standard solution from each | injection of standard solution. |
| | | parameter and each | The percentage relative standard |
| | | robustness condition was | deviation (% RSD) of area counts of |
| | | found within the acceptance | Sovateltide peak from three replicate |
| | | criteria. | injections of standard solution should not |
| | | The % relative standard | be more than 10.0%. |
| | | deviation of area counts of | |
| | | D-His peak from six | |
| | | replicate injections of | |
| | | standard solution from each | |
| | | parameter and each | |
| | | robustness condition was | |
| | | found within the acceptance | |
| | | criteria. | |

Although the field of the invention has been described herein with limited reference to specific embodiments, this description is not meant to be construed in a limiting sense. Various modifications of the disclosed embodiments, as well as alternate embodiments of the invention, will become apparent to persons skilled in the art upon reference to the description of the invention.

1.-53. (canceled)

54. A lyophilized pharmaceutical composition comprising:
a) sovateltide;
b) about 20% to about 80% w/w trisodium citrate; and
c) about 20% to about 80% w/w mannitol;
wherein the composition comprises not more than 1.0% of D-His-sovateltide impurity.

55. The lyophilized pharmaceutical composition of claim 54, wherein the composition is a lyophilized powder.

56. The lyophilized pharmaceutical composition of claim 54, comprising about 0.01% to about 0.02% w/w of the sovateltide.

57. The lyophilized pharmaceutical composition of claim 54, comprising about 30 μg sovateltide, about 50 mg trisodium citrate, and about 160 mg mannitol.

58. The lyophilized pharmaceutical composition of claim 54, wherein the total impurities are not more than 2%.

59. The lyophilized pharmaceutical composition of claim 54, wherein any unspecified impurities are not more than 0.5%.

60. The lyophilized pharmaceutical composition of claim 54, further comprising sodium chloride.

61. A reconstituted liquid composition, comprising:
a) the lyophilized pharmaceutical composition of claim 54, and
b) a liquid, wherein the liquid is in an amount sufficient to reconstitute the lyophilized pharmaceutical composition,
wherein the reconstituted liquid composition comprises not more than 1.0% of D-His-sovateltide impurity.

62. The reconstituted liquid composition, of claim 61, wherein the liquid is water or a 0.9% sodium chloride solution.

63. The reconstituted liquid composition of claim 61, wherein the sovateltide is 6 μg/ml.

64. The reconstituted liquid composition of claim 61, wherein the composition has an osmolarity of 240 mOsm/L and 310 mOsm/L.

65. The reconstituted liquid composition of claim 61, wherein the reconstituted liquid composition has pH of about 7.0 to 8.5.

66. The reconstituted liquid composition of claim 61, wherein the total impurities in the reconstituted liquid composition are not more than 2%.

67. The reconstituted liquid composition of claim 61, wherein any unspecified impurities in the reconstituted liquid composition are not more than 0.5%.

68. The reconstituted liquid composition of claim 61, wherein the reconstituted liquid composition is suitable for intravenous administration.

69. A container comprising the lyophilized pharmaceutical composition of claim 54.

70. The container of claim 69, wherein the lyophilized pharmaceutical composition comprises about 30 μg sovateltide.

71. The container of claim 69, wherein the lyophilized pharmaceutical composition comprises about 50 mg trisodium citrate.

72. The container of claim 69, wherein the lyophilized pharmaceutical composition comprises about 160 mg mannitol.

73. The container of claim 69, further comprising water or a 0.9% sodium chloride solution.

74. The container of claim 69, wherein the container is a vial.
